(19) United States
(12) Patent Application Publication
(10) Pub. No.: US 2016/0097777 A1
(43) Pub. Date: Apr. 7, 2016

(54) IMMUNOLOGICAL METHOD

(71) Applicant: GYROS Patent AB, Uppsala (SE)

(72) Inventors: Cecilia Bill, Uppsala (SE); Ann-Charlott Steffen, Solna (SE); Mats Inganäs, Uppsala (SE)

(21) Appl. No.: 14/849,326

(22) Filed: Sep. 9, 2015

(30) Foreign Application Priority Data
Sep. 12, 2014 (SE) 1451060-6

(51) Int. Cl. G01N 33/68

(52) U.S. Cl. CPC G01N 33/6854

(57) ABSTRACT

The present invention relates to an in vitro method for the detection of an anti-drug antibody (ADA) against a therapeutic drug antibody (TDA) in a biological sample. The in vitro method comprises detecting and capturing said ADA using affinity moieties obtained from an intact therapeutic drug antibody, or a Fab fragment thereof, in a certain manner. Avoiding using only intact therapeutic drug antibodies or only Fab fragments thereof as reagents in such a method has been proven as an efficient way of increasing sensitivity, decreasing unspecific binding and the formation of large protein complexes. Such a method has great utility in therapeutic drug development or in the evaluation of a particular treatment of a patient.

### TECHNICAL FIELD OF THE INVENTION

The invention relates to the field of immunological assays and methods, and in particular methods involving the detection of an analyte, more specifically an anti-drug antibody (ADA), in a biological sample from a patient treated with a therapeutic drug antibody. The invention relates to the field of methods useful for deciding if a therapeutic drug antibody is capable of inducing adverse drug reactions or other severe immunological reactions in a patient treated therewith.

### BACKGROUND OF THE INVENTION

### Antibodies

Antibodies (Abs) or immunoglobulins (Ig) are large proteins produced by plasma cells as a way of neutralizing antigens to prevent infections. The most abundant class found in plasma is IgG which has a molecular weight (MW) of approximately 150 kilodaltons (kDa). There are two different types of polypeptide chains present in IgG, the heavy chain of 50 kDa and the light chain of 25 kDa. IgG is composed of two heavy chains linked together by two disulfide bonds and two light chains linked to each heavy chain by a disulfide bond. IgG can be cleaved into two types of fragments with different functions. The first fragment is involved in antigen binding and is therefore called Fragment antigen binding (Fab). Each IgG contain two identical Fab fragments. The other fragment is involved in communicating with other parts of the immune system. When first discovered it was notably easy crystalized and therefore it is called Fragment crystallizable (Fc). Each IgG contain one Fc fragment. The part of an IgG where the two Fab fragments are connected to the Fc fragment is called the hinge region.

By an specific enzymatic reaction below the hinge region IgG can be digested into F(ab′)2 fragments. F(ab′)2 fragments can be further reduced into Fab fragments by dithiothreitol (DTT) (Lee et al. 2005). DTT reduces disulfide bonds into two thiol groups (Drugbank 2014). One method for evaluation of digestion and reduction procedures of IgG is sodium dodecyl sulfate polyacrylamide gel electrophoresis (SDS-PAGE).

### Monoclonal Antibodies

In 1975 Köhler & Milstein published a technology which enabled the production of murine (derived from mouse) mAbs (monoclonal antibodies). mAbs are Abs produced by a single clone of plasma cell, meaning they have identical structure including their antigen-binding site and therefore react with the same antigenic epitope. After the production of murine mAbs technical advances soon allowed chimeric, humanized and finally fully human mAbs to be produced. Chimeric Abs have murine variable parts and human constant parts. Humanized Abs differs from chimeric Abs in the variable parts which are composed of both human and murine parts.

### Monoclonal Antibodies as Therapies, Development of Anti-Drug Antibodies (ADA)

Today mAbs are used as targeting therapies for several diseases including autoimmune and infectious diseases, oncological disorders and transplant rejection (Hansel et al. 2010). In 2012, Reichert et al reported that 28 mAbs had been approved for the market and in 2013 (Reichert et al) that around 350 were in development. One advantage of using protein drugs, like mAbs, instead of low-molecular-mass drugs is their high target specificity. This feature increases treatment efficacy and selectivity. Another advantage is the long plasma half-lives of mAbs (up to 4 weeks) which reduce administration frequency (Keizer et al. 2010).

In 1998 Food and Drug Administration (US) approved Infliximab (IFX) with trade name Remicade for clinical use. IFX is a chimeric IgG1 mAb, has a MW of approximately 149.1 kDa and a half-life of 7.7 to 9.5 days (Food and Drug Administration 2014). IFX targets the naturally occurring human cytokine Tumor necrosis factor (TNF) by forming a stable complex which prevents TNF from binding to its receptor and thereby neutralizes its biological activity (Bao et al. 2014). The primary role of TNF is to regulate different components of the immune system. IFX is used as treatment for several autoimmune diseases like Crohn's disease, Psoriasis and Rheumatoid arthritis (RA) (Hansel et al. 2010). The recommended dose of IFX is 3-5 mg/kg every 8 weeks (Food and Drug Administration 2014).

One major concern when administrating protein drugs to patients is the risk for immunogenicity (Hansel et al. 2010). Immunogenicity is the patients' ability to induce an immune response towards the protein drug, meaning Abs directed against the drug are developed (Araujo et al. 2011). This immune response is called ADA response (Li et al. 2011) and like all human antibody responses a majority of ADA produced is of IgG class (Keizer et al. 2010).

To minimize ADA response therapeutic mAbs are often designed as chimeric, humanized or fully human but immune response may occur also towards fully human mAbs (Rispens et al. 2012). Patients' symptom caused by immunogenicity can differ from mild, for example, skin reactions at injection site, to adverse side effects like fatal allergic reactions (Hansel et al. 2010). Most importantly the effect of drug may be hampered or even eliminated by the appearance of ADA. Monitoring ADA response during clinical trials and potentially in routine use is therefore important (Li et al. 2011).

### Bridging Immunoassay—One Way of Detecting ADA—Influence of Interfering Serum Factors

An immunoassay is a method to quantitatively measure an unknown concentration of analytes. The analyte is measured using its specificity towards Abs (Cox et al. 2012). Monitoring ADA response is often conducted using a bridging immunoassay. A bridging immunoassay consists of three components, a capturing molecule, the analyte and a detecting molecule that has been labelled with a detectable compound, e.g. a fluorophore (Li et al. 2011). In a bridging immunoassay the capturing and detecting molecules are drug molecules, thus ADAs' bivalency, its ability to bind two antigens, can be utilized and the ADA molecule will form a bridge between capturing and detecting antigens (Tatarewicz et al. 2010).

One important parameter of ADA assay design is the CP value (Cut Point value) which is defined as the minimum level of response where a sample can be regarded as potentially positive for ADA (Wild 2013). Samples displaying response levels below CP is regarded as probably negative samples. To determine CP a data set of response values from untreated patients should be normally distributed and significant outliers should be identified and not included in the CP determination. To include possible differences in execution, a floating cut point (FCP) for each run should be calculated. The FCP is based on the CP from assay validation and a negative control sample analysed in every assay run (Shankar et al. 2008).

When developing an ADA assay the coefficient of variation (CV) is an important parameter for evaluation of assays robustness. An assays robustness is its capacity to be unaffected by small method variations (Shankar et al. 2008). Ligand Binding Assay Bioanalytical Focus Group recommends CV to be <20% for assays like bridging immunoassays (DeSilva et al. 2003).

In a mAb ADA assay there is a possibility that signal responses could be affected negatively by assay components. In theory optimal detection would occur when one capture reagent and one detection reagent are bridged by one ADA molecule. However, due to the bivalency of both labeled drug molecules and the ADA molecule there is a possibility that large complexes composed of several reagent and ADA molecules could form. Hypothetically, these large complexes would probably cause problems in detection or not interact with the immobilized streptavidin (forming the solid support) efficiently due to steric hindrance.

There is also a possibility that ADA molecules, due to its bivalency, will react with only one capture or detection reagent instead of bridging reagents hence forming undetectable incomplete complexes. Both scenarios, large complexes and incomplete complexes, lead to decreased signal response and may therefore cause false negative reactions.

Also, drug molecules remaining in patient circulation after dosing may interfere with ADA assays by complexing the ADA, not allowing it to be detected by the assay (Mikulskis et al. 2011). Therefore a functioning ADA assay must be drug tolerant. One way of making assays more drug tolerant is to dissociate ADA-drug complexes in samples using acid (Li et al. 2011).

When designing an immunoassay many serum factors could interfere. One potentially interfering factor for bridging immunoassays is Rheumatoid Factors (RFs). RFs are autoantibodies often associated with RA and the prevalence in RA patients can be as high as 70-90%. Prevalence of RF in the normal population is very low, about 1-2% (Araujo et al. 2011). RFs are often of isotype IgM and bind epitopes on Fc fragments of IgG which results in complex formation. These complexes are found in joints of RA patients and can activate other parts of the immune system like the complement system which eventually will lead to tissue damage (Corper et al. 1997). In a bridging immunoassay the prevalence of RFs can interfere by bridging Fc portions of capture and detecting reagents. This will increase signal responses and therefore cause false positive reactions (Tatarewicz et al. 2010).

In addition, it has previously been shown that anti-IgG hinge autoantibodies can be found in serum of the human population. Anti-IgG hinge autoantibodies bind to the hinge region of a cleaved antibody (i.e. the Fab fragment) but not to the intact IgG counterpart (Brerski, Knight & Jordan 2011).

### SUMMARY AND PRIOR ART

As biological drugs, such as therapeutic drug antibodies, are increasingly being used and put on the market, there is a great need for improved methods for detecting early clinical adverse effects of such drugs to avoid severe complications for the patients that are being treated.

Several methods for the detection of anti-drug antibodies (so called ADA assays) are available today. However, there is a constant urge for improving assay sensitivity as well as reducing disturbance from biological factors influencing the assay signal intensity. Still, it has been shown that these objectives are difficult to achieve.

WO2012/022774 discloses an immunoassay for the determination of antibodies against anti-therapeutic monoclonal antibodies wherein a Fab fragment of a therapeutic drug antibody is used as a capture reagent, however the detection reagent is not based on a therapeutic drug antibody.

Kato et al. (1979) and Rispens et al. (2012) have described similar experiments using reagent Fab and F(ab′)₂ fragments in ELISA format with the purpose to minimize interference from Reumatoid Factor. Such an assay is still likely to be affected by negative factors shown herein to be present when using only Fab fragments of the therapeutic drug antibody as the basis for the capture and detection reagents.

It is still to be revealed how to construct an ADA assay which reduces the influence of biological factors on the signal response therefrom, and in addition which is less affected by other disturbances decreasing the sensitivity of the assay.

Accordingly, available methods still suffer from one or more of the above problems. Hence, there is still a need in the art for improved immunological assays for detecting anti-drug antibodies (ADA) in patients treated with therapeutic drug antibodies.

### SUMMARY OF THE INVENTION

The object of the present invention is to solve or at least mitigate the above mentioned problems with current immunological assays for detecting anti-drug antibodies.

This object is met by providing an in vitro method for the detection of an anti-drug antibody (ADA) against a therapeutic drug antibody (TDA) in a biological sample, said method comprising: capturing and attaching said ADA onto a solid phase via a capture reagent, said capture reagent comprising an affinity moiety for said ADA and a capturing moiety for attachment to a solid phase; and detecting ADA with a detection reagent, said detection reagent comprising an affinity moiety for said ADA and a detection moiety for detecting the presence of ADA in the sample, wherein said capture reagent and said detection reagent thereby forms a complex or a bridging complex with said ADA, wherein said affinity moiety of said capture reagent and said affinity moiety of said detection reagent are selected from the group consisting of an intact TDA and one or more Fab fragment(s) of said TDA; and a) when the affinity moiety of said capture reagent is an intact TDA, the affinity moiety of said detection reagent is one or more Fab fragment(s) of said TDA, and b) when the affinity moiety of said capture reagent (1) is one or more Fab fragment(s) of said intact TDA, the affinity moiety (2a) of said detection reagent is an intact TDA.

Another object of the invention is to provide a kit comprising means for performing the in vitro method presented herein, i.e. a detection reagent, a capture reagent of the type mentioned herein as well as other reagents suitable for performing the method. Optionally instructions for use are also provided with said kit.

Accordingly, an in vitro method, or an immunological assay as presented herein, utilizing a particular combination of the affinity moieties of the detection reagent and the capture reagent, avoids, or at least reduces, the risk of obtaining false positive results and eventual difficulties in setting an appropriate cut-off value due to the presence of biological factors, such as particularly Rheumatoid factor (RF) and anti-hinge autoantibodies in mammalian specimen, such as human serum. Surprisingly, using an intact therapeutic drug antibody as a capture reagent and a Fab fragment as a detection reagent, or the reversed, appears to be particularly suitable to avoid such disturbances. Results are further presented herein which supports this finding.

In addition, other protein complex formation, aggregation of capture and detection reagents etc. also appear to be avoided when using an in vitro method as presented herein thereby providing a more sensitive method with less background.

It is a further objective to provide the use of an in vitro method for the detection of an anti-drug antibody (ADA) against a therapeutic drug antibody (TDA) in a biological sample as presented herein, for determining if a patient treated with an therapeutic drug antibody has developed a sensitivity or an adverse drug reaction against said therapeutic drug antibody. Such a method may be useful for determining if a patient should switch to another treatment, or modify the current treatment.

It is a further objective of the invention to provide an in vitro method, for the detection of an anti-drug antibody (ADA) against a therapeutic drug antibody (TDA) in a biological sample as presented herein wherein the method is performed using a device comprising a biological sample inlet; a detection reagent inlet; a capture reagent inlet; and a mixing chamber in fluid contact with the sample inlet and the detection reagent inlet, wherein the mixing chamber is positioned in a direction downstream of the sample inlet and the detection reagent inlet; and a column comprising the solid phase, wherein the column is in fluid contact with the biological sample inlet, the detection reagent inlet, the capture reagent inlet, and the mixing chamber, and wherein the column is positioned in a direction downstream of the capture reagent inlet and the mixing chamber; and an outlet in fluid contact with the column, wherein the outlet is positioned in a direction downstream of the column; and a means for detecting the detection reagent.

### BRIEF DESCRIPTION OF THE FIGURES

FIGS. 1a to 1d show a schematic figure over different assay variants based on therapeutic drug molecules and fragments thereof as capture and detection reagents. a) Biotinylated intact antibody and fluorophore labeled Fab. b) Biotinylated Fab and fluorophore labeled Fab. c) Biotinylated Fab and fluorophore labeled intact antibody. d) Biotinylated intact antibody and fluorophore labeled intact antibody.

FIGS. 2a-d shows assay variants biotinylated Infliximab and fluorophore labeled Infliximab, and biotinylated Fab and fluorophore labeled Fab evaluated in four equimolar reagent concentrations (defined in binding sites) in Gyrolab™. Standard points, 125-4000 ng/mL anti-Infliximab diluted in neat serum were measured in triplicates. Standard points were diluted 1:10 in Rexxip™ ADA before analysis on Gyrolab Bioaffy™ 200. a) Fab assay 13.3 nM and Infliximab assay 6.7 nM. b) Fab assay 40 nM and Infliximab assay 20 nM. c) Fab assay 120 nM and Infliximab assay 60 nM. d) Fab assay 360 nM and Infliximab assay 180 nM.

FIG. 3 shows the assay variants biotinylated Infliximab and fluorophore labeled Infliximab, and biotinylated Fab and fluorophore labeled Fab evaluated in Gyrolab™ using different diluents. 40 nM biotinylated Fab, 40 nM fluorophore labeled Fab, 20 nM biotinylated Infliximab and 20 nM fluorophore labeled Infliximab were used. Standard points, 125-4000 ng/mL anti-Infliximab diluted in neat serum or Rexxip™ ADA, were measured in triplicates and blank samples, in neat serum or Rexxip™ ADA, were measured in 6-plicate. Standard points and blank samples were diluted 1:10 in Rexxip™ ADA before analysis on Gyrolab Bioaffy™ 200.

FIG. 4 illustrates signal intensity and assay sensitivity of the assay variants presented in FIG. 1a-d. The assay variants biotinylated Infliximab and fluorophore labeled Fab (b-IFX, f-Fab, square), biotinylated Fab and fluorophore labeled Fab (b-Fab, f-Fab, plus), biotinylated Fab and fluorophore labeled Infliximab (b-Fab, f-IFX, circles), and biotinylated Infliximab and fluorophore labeled Infliximab (b-IFX, f-IFX, cross) were evaluated in Gyrolab™. 40 nM biotinylated Fab, 40 nM fluorophore labeled Fab, 20 nM biotinylated Infliximab and 20 nM fluorophore labeled Infliximab were used. Standard points, 125-4000 ng/mL anti-Infliximab diluted in neat serum were measured in triplicates and blank samples diluted in neat serum were measured in 6-plicate. Standard points and blank samples were diluted 1:10 in Rexxip™ ADA before analysis on Gyrolab Bioaffy™ 200.

FIGS. 5a and 5b show a scatter plot illustrating average response values from anti-drug antibody (ADA) assay in screened serum samples; blood donor samples (circles), IFX patients, i.e. patients treated with therapeutic drug Infliximab (diamonds), RF patients i.e. patients positive for Rheumatoid factor (plus). The assay variant used is illustrated in FIG. 1d (b-IFX, f-IFX). FIG. 5a) and b) are results from the same assay variant and run, but in b) the scale on the y axis is more concentrated around the cut point (cut point is shown as dashed line in FIG. 5b)). In FIG. 5b) the four highest positive response values for the IFX-patients are not shown.

FIGS. 6a and 6b shows a scatter plot illustrating average response values from anti-drug antibody (ADA) assay in screened serum samples; blood donor samples (circles), IFX patients, i.e. patients treated with therapeutic drug Infliximab (diamonds), and RF patients i.e. patients positive for Rheumatoid factor (plus). The assay variant used is illustrated in FIG. 1a; (b-IFX and f-fab). FIGS. 6a) and 6b) are results from the same assay variant and run, but in b) the scale on the y axis is more concentrated around the cut point (cut point is shown as dashed line in FIG. 6b)). In FIG. 6b) the four highest positive response values for the IFX-patients are not shown.

FIG. 7 shows a scatter plot illustrating average response values from anti-drug antibody (ADA) assay in screened serum samples; blood donor samples (circles), and patients positive for rheumatoid factor (plus). The assay variant used is illustrated in FIG. 1b)(b-Fab, f-Fab).

FIGS. 8a-8d shows the four assay variants in FIG. 1a-d evaluated in terms of drug tolerance in Gyrolab™. 40 nM biotinylated Fab, 40 nM fluorophore labeled Fab, 20 nM biotinylated Infliximab and 20 nM fluorophore labeled Infliximab were used. Standard points, 125-4000 ng/mL anti-Infliximab diluted in neat serum spiked with free drug were measured in triplicates and blank samples containing neat serum spiked with drug were measured in 6-plicate. Standard points and blank samples were diluted 1:10 in Rexxip™ ADA before analysis on Gyrolab™ Mixing CD with acid dissociation. a) Biotinylated Fab and fluorophore labeled Fab. b) Biotinylated Fab and fluorophore labeled Infliximab. c) Biotinylated Infliximab and fluorophore labeled Fab. d) Biotinylated Infliximab and fluorophore labeled Infliximab.

FIGS. 9a-b shows examples of Gyrolab™ microfluidic structures. FIG. 9a shows a bioaffy structure (Gyros AB); a) Common channel; b) Hydrophobic barriers; c) Capture column pre-packed with streptavidin-coated beads; d) Individual inlet; FIG. 9b shows a mixing CD structure (Gyros AB); a) Inlet for samples/reagents/buffers; b) Mixing chamber; c) Capture column pre-packed with streptavidin-coated beads; d) Hydrophobic barriers.

FIG. 10 shows the effects of acid dissociation for assay variant biotinylated Fab and fluorophore labeled Fab investigated in Gyrolab™. 40 nM biotinylated Fab and 40 nM fluorophore labeled Fab were used. Standard points, 125-4000 ng/mL anti-Infliximab diluted in neat serum spiked with 0-10 μg/mL free drug were measured in triplicates. Standard points were diluted 1:10 in Rexxip™ ADA before analysis on Gyrolab™ Mixing CD with acid and neutral dissociation.

FIG. 11 shows a column profile for sample containing only fluorophore labeled Fab in 40 nM, the sample was analysed in Gyrolab™. Intensity 0-0.01 on y-axis.

FIG. 12 shows a column profile for sample containing only fluorophore labeled Infliximab in 20 nM, sample was analysed in Gyrolab™. Intensity 0-0.01 on y-axis.

FIG. 13 shows results from SDS-PAGE when digestion of Infliximab was evaluated. Reduced Infliximab is visible in lane 1, non-reduced Infliximab in lane 2, reduced F(ab′)2 is visible in lane 3, non-reduced F(ab′)2 is visible in lane 4 and the reference sample is visible in lane 5.

FIG. 14 shows results from SDS-PAGE when reduction of Infliximab F(ab′)2 was evaluated. Reduced Fab is visible in lane 1, non-reduced Fab is visible in lane 2, reduced F(ab′)2 is visible in lane 3, non-reduced IFX is visible in lane 4 and the reference sample is visible in lane 5.

### DETAILED DESCRIPTION OF THE INVENTION

### Definitions

The in vitro method may also be referred to herein as an “assay” or an “immunoassay”. There are several possible assay formats which are utilizable for an in vitro method of the invention, of which examples are further described herein. The term “immunoassay”, which is well known in the art, refers to a specific binding assay in which an analyte, e.g. ADA, is detected by use of at least one antibody as a reagent.

The term “antibody” (immunoglobulin) should be interpreted broadly herein and refers to monoclonal antibodies, polyclonal antibodies as well as fragments thereof. The antibodies are divided into classes and include IgA, IgD, IgE, IgM and subclasses thereof, such as IgG1, IgG2 etc.

The “Fab fragment” (fragment antigen binding) as defined herein, contains the variable regions of the light chain and the heavy chain, respectively, as well as the constant domain of the light chain and the first constant domain of the heavy chain (CH1). Each IgG contain two identical Fab fragments F(ab′)₂. However, when a “Fab fragment” is referred to herein this may refer to either a monovalent Fab or to a bivalent F(ab′)₂. F(ab′)₂ is obtained from the intact antibody molecule by cleavage at the hinge region. The bivalent F(ab′)₂ molecule may then be further reduced into two monovalent Fab's. In addition, a Fab fragment used in an in vitro method herein may further comprise additional amino acids in one or more ends of the protein or peptide if this is suitable for design purposes.

“Bivalent” or “Bivalency” as used herein refers e.g. to a Fab fragment (antibody fragment) of an intact antibody having two binding sites, for an antigen/epitope, or to an intact antibody having two binding sites for an antigen/epitope. F(ab′)₂ is an example of a bivalent Fab fragment. A “monovalent” Fab fragment refers to a Fab fragment having one binding site for an antigen/epitope, herein exemplified by one arm of the F(ab′)₂ region.

An “intact” antibody as used herein, refers to an intact antibody molecule including the Fab (fragment antigen binding) region/fragment, the Fc (fragment crystallizable) region and the hinge region of the antibody. The antibody is intact in the meaning that it has not been cleaved to generate separate antibody fragments, nor have any parts relevant for its function been removed from the antibody. The intact antibody used in a method herein may still have been slightly structurally modified while still maintaining its function.

An “anti-drug antibody”, “ADA”, or “ADA molecule” as used herein, refers to an antibody which binds to a therapeutic drug antibody, i.e. an antibody that has affinity for a therapeutic drug antibody binding to said therapeutic antibody thereby risking interfering with the therapeutic activity thereof. An ADA is the result of an adverse drug reaction in a patient treated with a therapeutic drug antibody. An ADA may bind to a therapeutic drug forming large immune complexes which can make it difficult to detect the presence of a therapeutic drug in a treated patient. In the in vitro method according to the present invention, the ADA is the “analyte” often referred to in the context of an immunoassay.

A “capture reagent” as used herein, refers to an agent or a reagent which has affinity for an analyte in a sample which is to be analyzed, i.e. the ADA, and can bind to a solid phase or a solid support. Therefore, the capture reagent can be defined as having two parts, one affinity moiety and one capturing moiety where the affinity moiety has affinity sites for, and binds to ADA, and the capturing moiety has capturing capabilities making it possible to bind or attach, e.g. covalently, through its capturing moiety to reactive sites of a solid phase. The affinity moiety of a capture reagent in the context of the present invention, comprises an intact therapeutic drug antibody or one or more Fab fragments thereof.

A “detection reagent” as used herein refers to an agent or a reagent which provides for affinity binding to an analyte in a sample which is to be analyzed, i.e. the ADA, and for detection and/or quantification of the presence of said analyte in a sample. The detection reagent can hence be defined as having two parts, one affinity moiety having affinity sites for, and binding to, ADA, and one detection moiety providing for the detection, such as optical detection, and/or quantification of ADA in a biological sample. The detection moiety may include a label, as further described herein. The affinity moiety of the detection reagent in the context of the present invention comprises an intact therapeutic drug antibody or one or more Fab fragments thereof. Herein, the term “detection” can be used broadly referring both to qualitative and quantitative measurements of an analyte. Accordingly, detection may be performed to detect mere presence and/or to quantify the amount of ADA in a sample.

The term “label” as used herein, and optionally forming a part of the detection reagent, refers to any substance that is capable of producing a detectable signal, whether visibly (optically) or by using suitable instrumentation.

The term “affinity moiety”, as used herein, refers to a part of the capture reagent or the detection reagent which is arranged with affinity sites for the anti-drug antibody, ADA, meaning that the moiety will interact and bind to ADA, allowing for further detection and quantification thereof. The sites of the affinity moiety that are responsible for the affinity binding to ADA comprise the antigen binding sites of the Fab fragment of the therapeutic drug antibody or comprises only the Fab fragment thereof when the Fab fragment is used as a detection or a capture reagent. The antigen binding sites are specific for the particular ADA that is to be detected in the in vitro method. A specific binding is intended to mean that binding is to a particular peptide without substantial binding to another peptide (also referred to as an epitope).

A “capturing moiety” as used to herein, refers to a part of the capture reagent of an in vitro method as presented herein which provides for the attachment of the capture reagent to the solid phase or the solid support. An example of a capturing moiety is a biotin molecule.

A “solid support” or a “solid phase” refers to an insoluble, functionalized, polymeric material to which reagents may be attached or covalently bound (often via a linker) to be immobilized or allowing them to be readily separated (e.g. by filtration, centrifugation, washing etc.) from excess reagents, soluble reaction by-products, or solvents. Solid phases or supports for use in the present invention are known in the art, and examples are also provided herein.

When a “bridging complex” or a “complex” is referred to herein, this is intended to mean a complex formed between the capture reagent, the ADA and the detection reagent, wherein the affinity moieties of the respective detection and capture reagent are responsible for the binding to both arms of the ADA molecule.

The term “therapeutic drug antibody”, “TDA” or “therapeutic drug” refers herein to an antibody which has been approved as a therapeutic and which may be administered to a patient for the treatment of a disease or a disorder. The therapeutic drug antibody may be a monoclonal antibody. When a TDA is referred to, this may also encompass an analogue or a structurally similar variant of said TDA, as long as the relevant function thereof is maintained. A therapeutic drug antibody particularly useful for a method as presented herein may be defined as an intact therapeutic drug antibody of any IgG sub class which may be cleaved to Fab fragments for subsequent use in a method as defined herein.

A “chimeric” antibody is an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

A “humanized antibody” is an antibody from a non-human species whose protein sequences have been modified to increase their similarity to antibody variants produced naturally in humans.

A “biological sample” as used herein refers to a sample, such as a clinical sample previously obtained from a patient or a subject, such as a human patient, which may for example be whole blood, serum, or plasma. The biological sample may comprise antibodies recovered from the patient, such as ADA, if the patient has been treated with a therapeutic drug antibody. The sample is thereafter used in an in vitro method as described herein. The biological sample may be taken from a patient before or after one or more treatments with a therapeutic drug antibody. A mammal may be a human patient, or a non-human mammal. A “reference” biological sample is a sample taken e.g. from an untreated patient and/or a healthy individual for comparison purposes.

A “cut point” is a statistically determined level above which samples are qualified as potentially positive responses, and below which level samples are considered as negative responses for determining the presence or non-presence of an analyte in a biological sample in an in vitro method according to the invention. Examples of determinations of cut point values and tools therefore are provided in the experimental section.

Used herein in addition to the particulars provided in the experimental section may be conventional techniques of molecular biology such as microbiology, cell biology, biochemistry, and immunology, which are within the ordinary skill of the art. Such techniques are presented in the literature, such as in, “Molecular Cloning: A Laboratory Manual”, second edition (Sambrook et al, 1989).

If not defined in any other manner technical and scientific terms used herein have the same meaning as commonly understood by one of ordinary skill in the art to which this invention belongs. However, herein, such known technical and scientific terms may also be complemented by further explanations of their meaning.

### DETAILED DESCRIPTION

One major concern when administrating protein drugs to patients is the risk for immunogenicity. The effect of drug may be hampered or even eliminated by the appearance of ADA (anti-drug antibodies). Hence, monitoring ADA response during clinical trials and potentially in routine use is therefore important.

There are several problems with assays seeking to determine the presence of ADA today, such as drug molecules remaining in patient circulation which may interfere by complexing the ADA molecule, as well as the formation of large or incomplete complexes including drug reagents and ADA molecules. In addition, and more importantly, other biological factors present in the serum of the patients have been shown to interfere with the ADA assay signal thereby providing false positive results.

Initially, one object herein was to improve ADA assay performance in general, and exemplified herein by the usage of Gyrolab™ workstation, for therapeutic drug antibodies (TDA).

When developing ADA assays in a bridging immunoassay format, several factors can interfere with a successful outcome thereof affecting both the sensitivity and the signal intensity of the assay.

ADA assay performance in Gyrolab™ is limited mainly by three factors which may inappropriately affect signal intensity levels. These effects are also applicable to other immunological techniques, and hence in general also need to be avoided when developing an immunological assay, in addition to factors affecting sensitivity of the assay.

Herein, the initial focus was on the following factors: —False negative reactions of assays when using bivalent intact therapeutic drug antibodies as both capture and detection reagents.

These reactions are hypothetically caused by large or incomplete complex formation between the bivalent capture reagent, detection reagent and ADA molecule thereby hiding the presence of ADA present in the biological sample and leading to decreased signal intensity; —False positive reactions which may be caused by interference of RF (Rheumathoid factor) with the Fc part of the intact therapeutic drug antibody. This reaction also leads to increased signal intensity; —Decreased assay sensitivity which hypothetically is caused by aggregation between capture and detection drugs. This reaction leads to increased background.

Hence, a bridging immunoassay (ADA) based on drug Fab fragments instead of intact drug was developed for the monoclonal antibody Infliximab to avoid the above drawbacks most commonly associated with the use of intact drug molecules as ADA assay reagents and with the purpose to minimize inappropriate effects on signal intensity.

During the development different assay variants were compared in terms of assay sensitivity and signal intensity to an assay based on intact drug. Cut point (CP) was determined for the most optimal assay variant based on samples from healthy blood donors and thereafter used for screening samples from patients treated with drug and samples tested positive for Rheumatoid factor (RF).

It was shown herein that an assay based entirely on an intact therapeutic drug (TDA) is more affected by false negative reactions due to large complex formation between the bivalent capture and detection reagents and the ADA molecule compared to an assay based on only drug Fab fragments. The assay based entirely on an intact therapeutic drug (TDA) was also shown to be affected by false positive results most likely due to the presence of Rheumatoid factor.

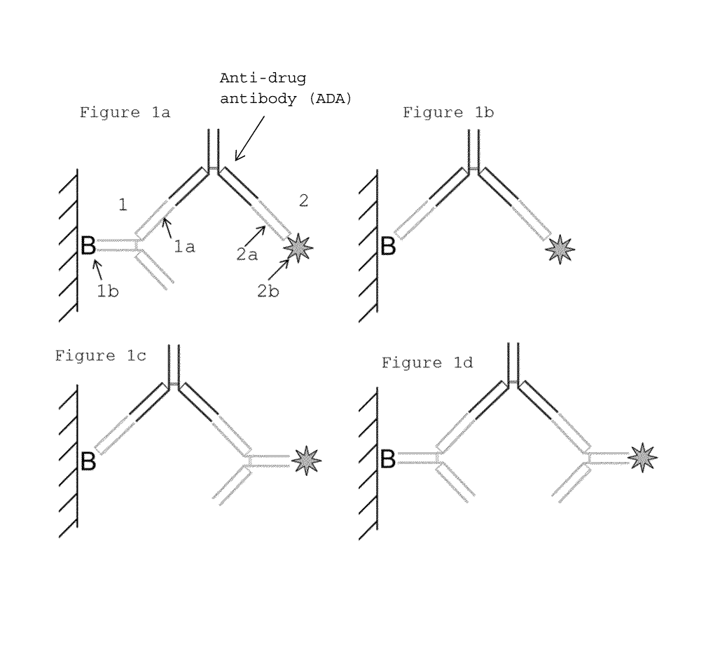

However, evaluating the variants illustrated in FIGS. 1a) to d) it was further surprisingly found that using a combination of capture reagent and detection reagent based on an intact therapeutic drug antibody and a Fab fragment thereof, respectively and vice versa, also substantially avoids false positive reactions of assays based only on Fab fragments of therapeutic drug antibodies in addition to the false positives of the assays based on the intact drug antibody.

Herein, it is without wishing to be bound by theory, envisaged that such false positive reactions in the Fab/Fab assay are due to the presence of anti-hinge autoantibodies in the serum of all individuals which bind and complex the Fab reagents. By using a combination of Fab and intact therapeutic drug antibodies as explained herein, the negative influence of both anti-hinge autoantibodies and RF has been shown to be avoided or at least mitigated. Hence, despite unpredictability within the art, this combination appears to significantly reduce the influence of these biological factors in an ADA assay setting, which has not previously been shown.

It is also shown herein that anti-hinge autoantibodies play a more important role than previously known when performing and analyzing the results from an ADA assay which need to be taken into account when setting the cut off value.

In addition, and surprisingly, the assay intact/Fab (i.e. where the capture reagent is and intact therapeutic drug antibody and the detection reagent is a Fab fragment thereof, more particularly a monovalent Fab fragment thereof) was also shown to overcome or at least mitigate the remaining drawbacks influencing the sensitivity of the method, such as protein complex formation and aggregation of capture and detection reagents.

Accordingly, the present inventors have found an immunological in vitro method as presented herein which alleviates the limitations to previous methods in the art.

The usage of such an in vitro method forms an important tool in therapeutic drug development as well as in the evaluation of patient treatment with therapeutic drug antibodies.

The method may be executed on a Gyrolab™ workstation (Gyros AB) but is also generally applicable to other immunological techniques providing appropriate technical support for the method presented herein. Gyrolab™ is an automated platform which can be used to develop bridging immunoassays where the anti-drug antibodies affinity towards the monoclonal antibody is utilized.

Accordingly, there is provided herein an in vitro method for the detection of an anti-drug antibody (ADA) against a therapeutic drug antibody (TDA) in a biological sample, said method comprising the steps of: capturing and attaching said ADA onto a solid phase via a capture reagent (1), said capture reagent comprising an affinity moiety (1a) for said ADA and a capturing moiety (1b) for attachment to a solid phase; and detecting ADA with a detection reagent (2), said detection reagent comprising an affinity moiety (2a) for said ADA and a detection moiety (2b) for detecting the presence of ADA in the sample, wherein said capture reagent (1) and said detection reagent (2) thereby forms a bridging complex with said ADA, wherein the method is characterized in that said affinity moiety (1a) of said capture reagent (1) and said affinity moiety (2a) of said detection reagent (2) are selected from the group consisting of an intact TDA and one or more Fab fragment(s) of said TDA; and a) when the affinity moiety (1a) of said capture reagent (1) is an intact TDA, the affinity moiety (2a) of said detection reagent (2) is one or more Fab fragment(s) of said TDA, and b) when the affinity moiety (1a) of said capture reagent (1) is one or more Fab fragment(s) of said intact TDA, the affinity moiety (2a) of said detection reagent (2) is an intact TDA.

Usually, the at least one Fab fragment used in the method herein is a monovalent Fab, i.e. the originally bivalent Fab fragment F(ab′)₂ of a therapeutic drug antibody has been cleaved into two Fab fragments of which one is used as an affinity moiety (2a) in a detection reagent (2) or as an affinity moiety (1a) of a capture reagent (1) in a method disclosed herein. This is further illustrated in FIG. 1.

Hence, the method according the invention utilizes a therapeutic drug antibody as a starting point for both the affinity moiety (1a) of the capture reagent (1) and the affinity moiety (2a) of the detection reagent (2) used in the present method. In a method as disclosed herein, an intact therapeutic drug antibody is however not used both for the capture reagent (1) and detection reagent (2), and neither is a Fab fragment thereof used both for the capture reagent (1) and the detection reagent (2). Instead a Fab fragment thereof is used for the capture reagent (1) or for the detection reagent (2), depending on the usage of the intact therapeutic drug antibody. The intact therapeutic drug antibody is cleaved into a F(ab′)₂ fragment and optionally further into a monovalent Fab to obtain a Fab fragment for use in the method. This is exemplified in the experimental section.

It is to be understood that the same therapeutic drug antibody, or an analogue, or a structurally similar variant thereof providing the same function, towards which ADA's are to be detected, forms the basis for the construction of the capture and the detection reagents in such a method set up.

Accordingly, the following combinations of affinity moieties (1a, 2a) are presented:

### Immunoassay 1:

Capture reagent (affinity moiety (1b))): Intact therapeutic drug antibody; Detection reagent (affinity moiety (2b)): Fab fragment of said therapeutic drug antibody. This version is also referred to herein as Intact/Fab version.

### Immunoassay 2:

Capture reagent (affinity moiety (1b))): Fab fragment of said therapeutic drug antibody;

Detection reagent (affinity moiety (2b)): Intact therapeutic drug antibody. This version is also referred to herein as Fab/intact version.

The sensitivity appears to be most improved when the combination of an intact therapeutic drug antibody is used as the affinity moiety of the capture reagent and the Fab fragment is used as the affinity moiety of the detection reagent (immunoassay 1). This variant is the most sensitive assay and appears not to be affected by false positive reactions caused by Rheumatoid factor or anti-hinge autoantibodies.

As explained herein, the reduced influence of Rheumatoid factor and anti-hinge autoantibodies on the signal response, and also on the sensitivity is most likely due to the specific combination of an intact therapeutic drug antibody and a Fab fragment thereof for the capture and detection reagents as explained herein. The further improvement of the sensitivity of the Intact/Fab assay may also be due to other structural details of the assay set up, such as the model system used (Infliximab).

Hence, it is envisaged that the influence by RA and anti-hinge autoantibodies is still mitigated by the combination of the specific capture and detection reagents as explained herein. The in vitro method herein is only exemplified by the usage of the model system Infliximab, but is not limited thereto.

FIG. 1 illustrates the different assay variants tested of which immunoassay 1 is illustrated in FIG. 1a and Immunoassay 2 in FIG. 1c.

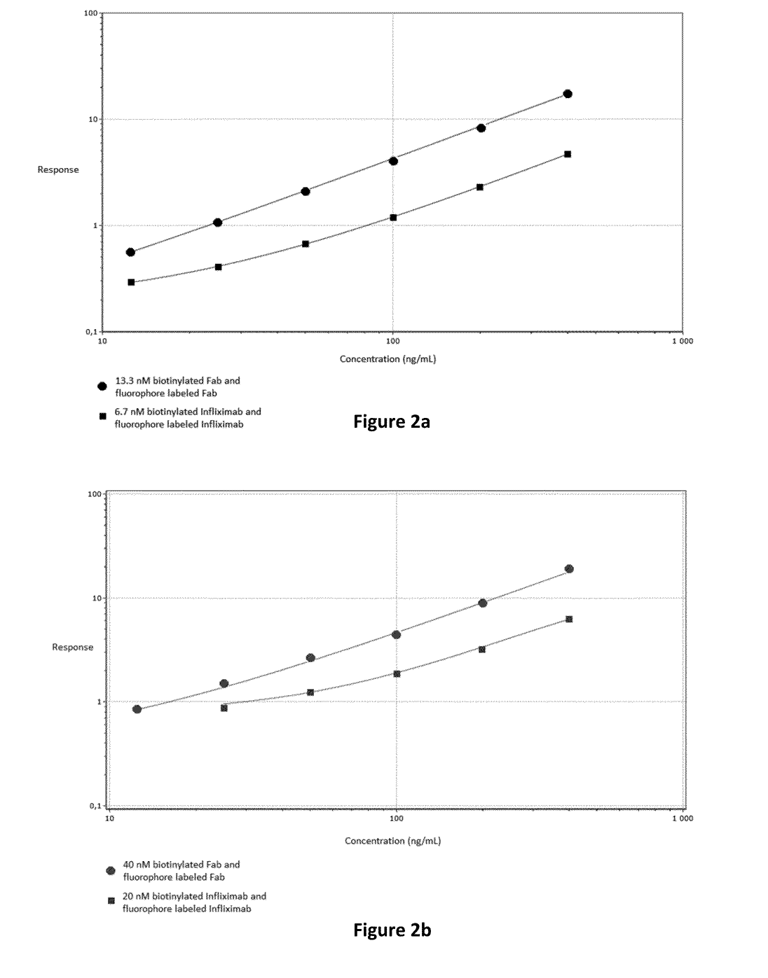

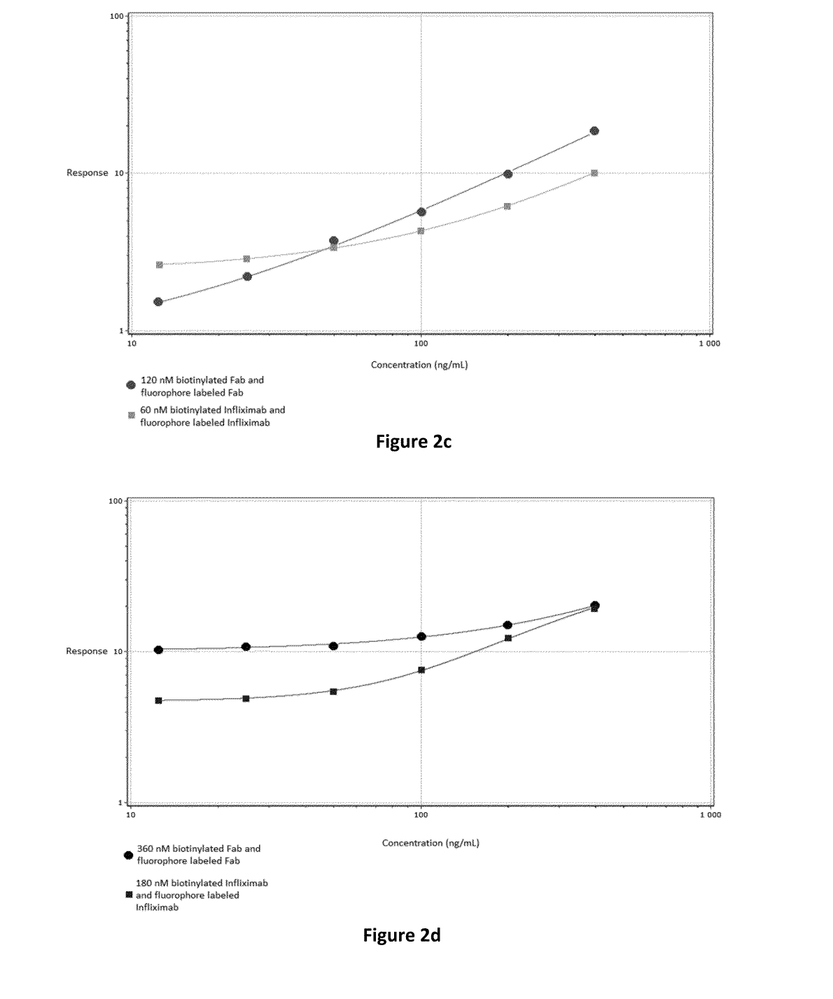

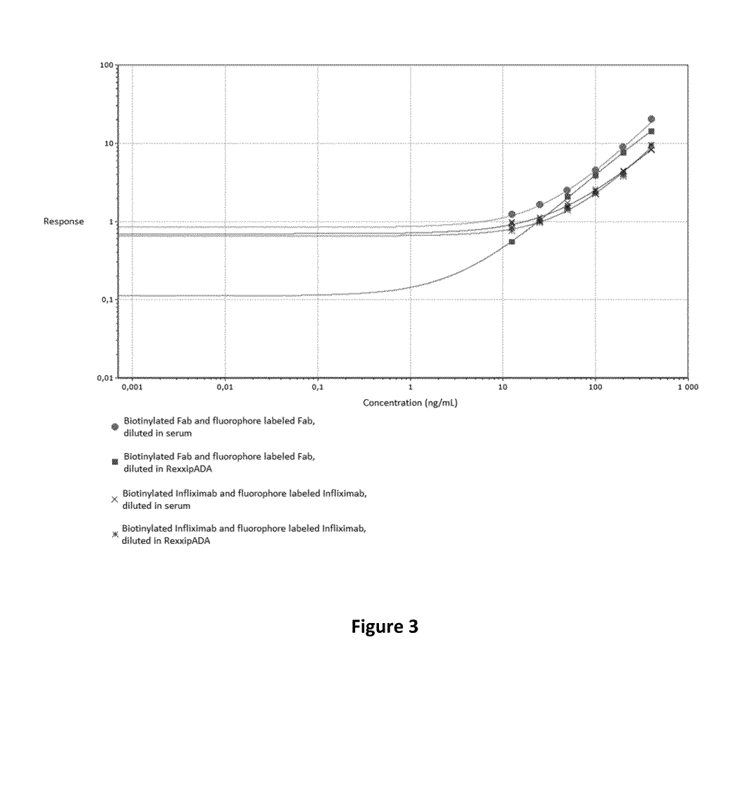

FIGS. 2-4 shows the result of assay sensitivity and signal intensity of the tested assays. FIGS. 2a-d show that the Fab/Fab assay generates higher signal response and lower background response than the assay based on the intact therapeutic drug antibody in all reagent concentrations when diluted in Rexxip™ ADA. FIG. 3 shows the result from Fab/Fab and intact/intact in Rexxip™ ADA dilution and neat serum dilution. Therein, Fab/Fab assay displayed higher signal response than Intact/Intact assay in both serum and Rexxip™ ADA. When Fab/Fab assay is diluted in serum, the background response is increased 10 times compared to dilution in Rexxip™ ADA, potentially due to interference from anti-hinge autoantibodies which complexes capture and detection reagents. This e.g. hinders the setting of an accurate cut-off value making it difficult to identify true positives only. Hence, utilizing a method which does not suffer from these problems may increase assay sensitivity and reduce problems with the signal.

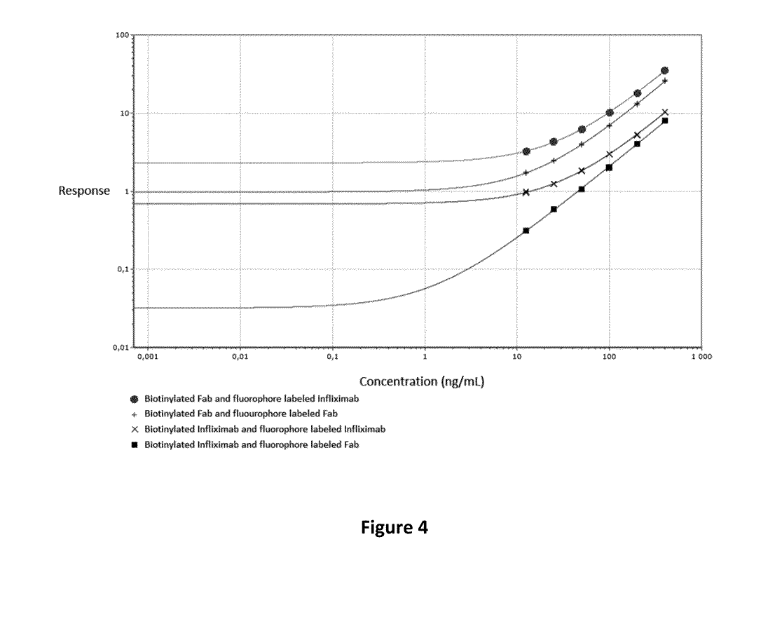

FIG. 4 shows the result from the investigation of the assay sensitivity and signal intensity in all assay variants. The assay variant intact/Fab results in the lowest background response and lowest signal response. The assay variant intact/Fab also display the best response dynamics. The high background response of the Fab/intact variant may be due to various background responses such as the formation of complexes or interactions with the solid support, potentially due to the model system that is being used.

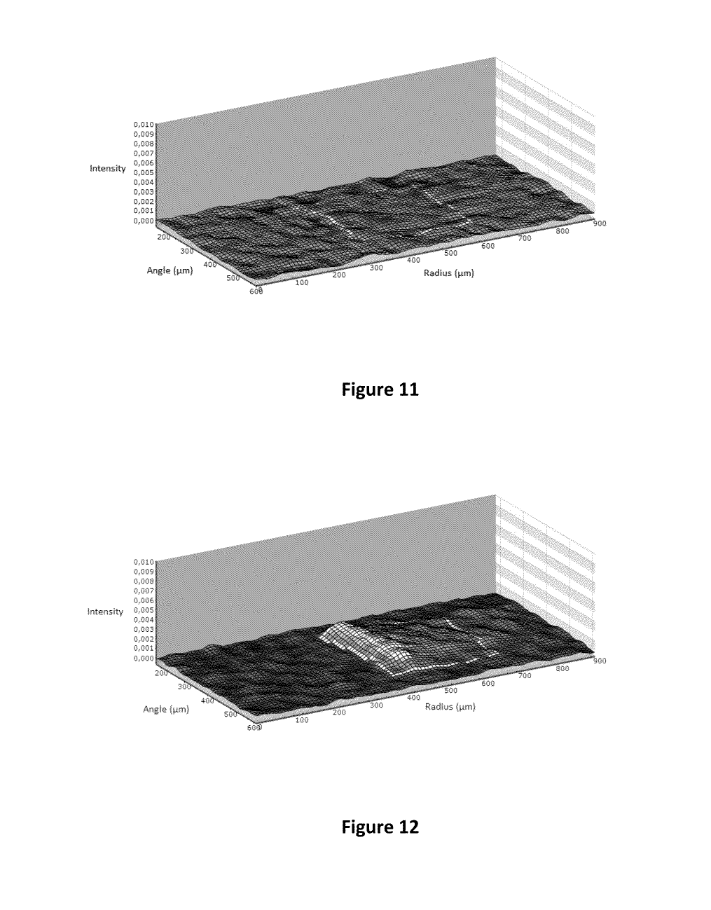

An experiment investigating background noise from Fab and Intact therapeutic antibodies as reagents has been performed and the results are shown in FIGS. 11 and 12. Therein it is shown that the intact therapeutic drug antibody is more prone to generate background, e.g. due to unspecific binding of the Fc part to the streptavidin column affecting the sensitivity of the assay. It is still envisaged that the Fab/intact version of the assay avoids substantial disturbance of the assay signal due to the lack of interaction with RF and anti-hinge autoantibodies.

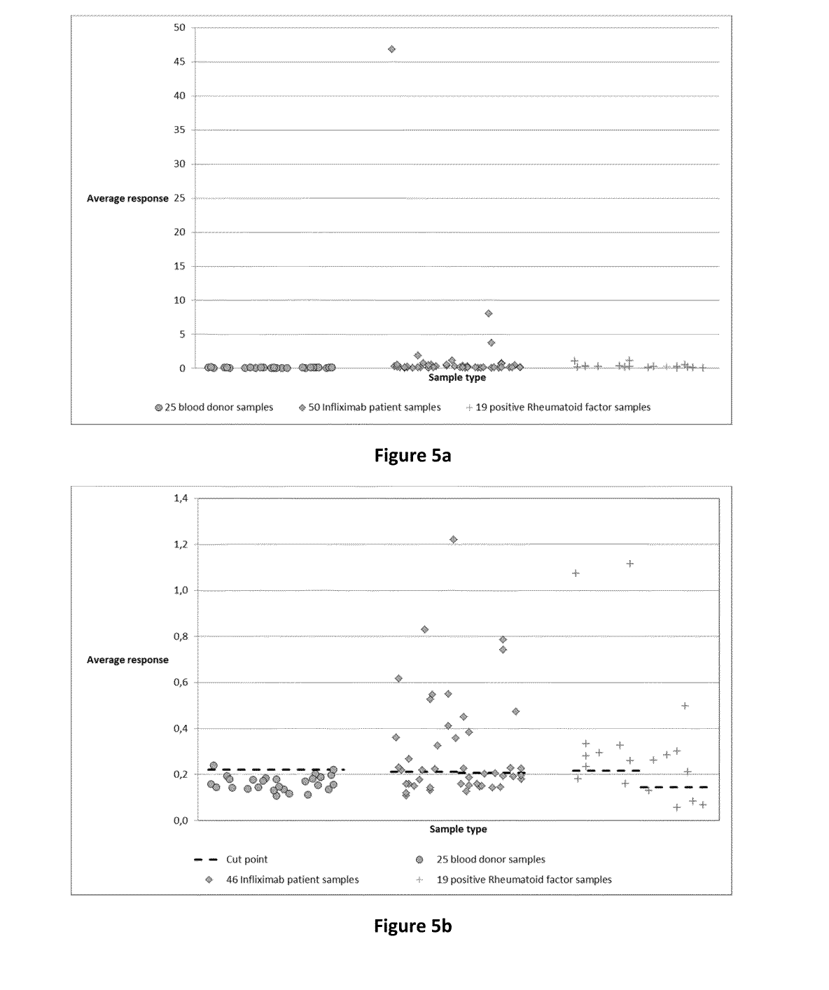

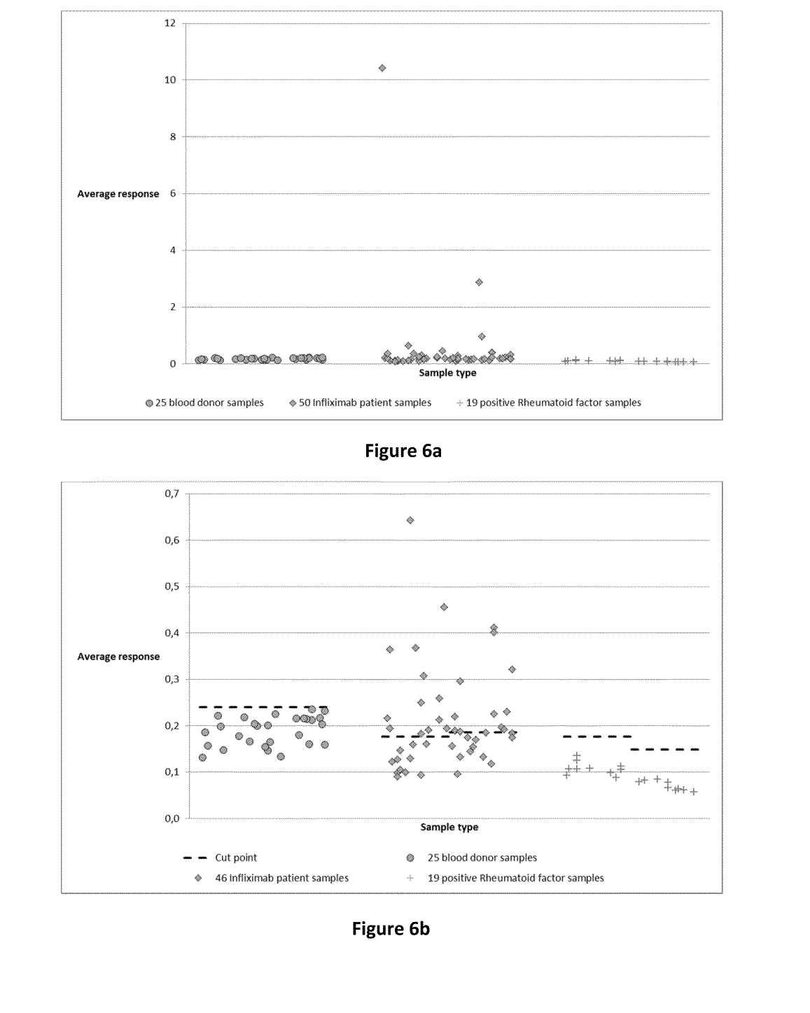

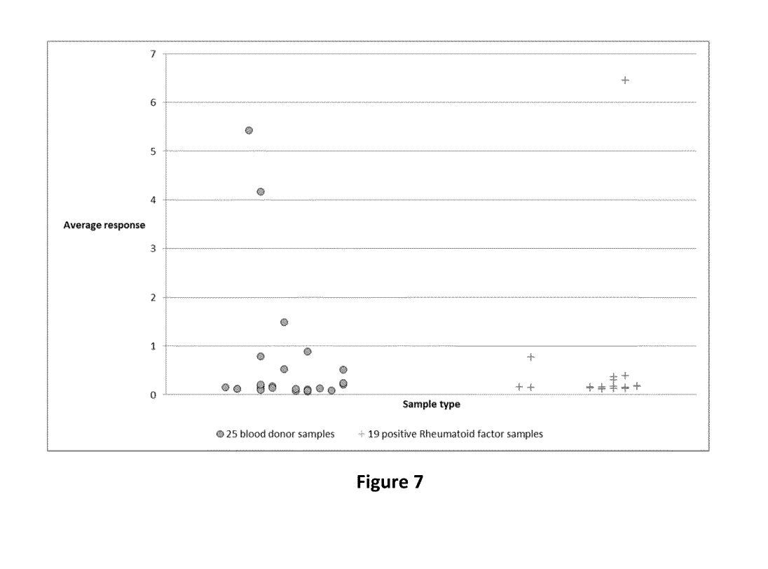

As shown in FIGS. 6a and 6b, illustrating the response levels in blood donors, IFX (Infliximab) patients and RF patients, the assay variant using intact therapeutic drug antibody as the affinity moiety (1a) of the capture reagent (1) and a Fab fragment as the affinity moiety (2b) of the detection reagent (2) generates no false positive results for the blood donors or for the RF patients. FIG. 6b shows the response values concentrated around the cut off value. This should be compared to the results presented in FIGS. 5a and 5b and 7, wherein the assay variants of FIGS. 1d) and 1b) were used, respectively.

The b-IFX/f-IFX variant (intact/intact) generates false positive results both in the RF patient group, but also in the blood donor group (FIG. 5). The b-Fab/f-Fab (Fab/Fab) variant also generates false positive results, particularly in the blood donor group.

Without wishing to be bound by theory, and as earlier mentioned herein it is envisaged that the false positive results in the Fab/Fab variant may partly be due to the presence of anti-hinge autoantibodies therein. If the anti-hinge autoantibodies bind to the exposed hinge region of the Fab fragments, these may form a bridge between the capture and the detection reagent and thereby form a complex which is detected as a (false) positive response. Further, the combination of intact/Fab avoids false positives which have previously been shown to appear in patients being positive for RF.

It was surprising to see that the combination of intact/Fab antibody effectively also avoids earlier drawbacks. Accordingly, the combination of Fab/intact or intact/Fab in the capture and detection reagents appears to avoid disturbance of the signal due to the presence of the biological factors, such as RF or anti-hinge autoantibodies.

Notable herein is that anti-hinge auto-antibodies seem to be frequent in a variety of populations, including healthy individuals. It has also been shown herein that these antibodies have a significant influence on the results of ADA assays. This finding is also useful for optimization of the cut-off value for the ADA assay allowing detection of as many true positives as possible. Setting the cut-off too high risks losing out on weak positive responses, on the other hand setting it too low previously risked to include false positive results. By at least avoiding including subjects only screening positive for RF or anti-hinge autoantibodies by using a method according to the invention the setting of the cut-off value may be further optimized allowing also true weak positives to be detected while excluding false positive reactions from anti-hinge autoantibodies and RF.

As earlier shown, the sensitivity of the assay also appears to be improved, particularly in the Intact/Fab variant providing an assay with less background e.g. caused by complex formation between capture and detection reaction with ADA as well as unspecific aggregation of the reagents.

Hence, presented herein is an in vitro method which solves or at least mitigates the earlier drawbacks of ADA assays by using a combination of an intact therapeutic drug antibody and a Fab fragment thereof, such as a monovalent Fab fragment thereof, thereby setting an immunological scenario which hinders the disturbance of negative factors on the results presented therefrom.

Accordingly, there is provided an in vitro method as defined herein wherein the affinity moiety (1a) of the capture reagent (1) is an intact TDA and the affinity moiety (2a) of the detection reagent (2) is one or more Fab fragment(s) of said intact TDA. There is further provided herein an in vitro method as defined herein wherein the affinity moiety (1a) of the capture reagent (1) is one or more Fab fragment(s) of said intact TDA and the affinity moiety (2a) of the detection reagent (2) is an intact TDA.

For detection of a bound ADA, the detection moiety (2b) of the detection reagent (2) may comprise a detectable label as further described herein. The capture moiety (1b) of the capture reagent (1) provides means for attaching the capture reagent to a solid phase or a solid support. The solid phase is provided with reactive sites for reacting with the capturing moiety of the detection reagent (2). Said solid phase may be saturated with the capture reagent (1) before the biological sample is provided thereto.

An immunological assay, or method, can be classified with respect to the number of incubations (steps) that are required to form the complex to be measured (in the present case a complex comprising an ADA molecule, detection reagent and capture reagent). Assay formats may be referred to a one-step formats when they are “simultaneous” (or “homogenous”), i.e. the analyte (ADA), the capture reagent and the detection reagent are reacted in the same incubation/step. Two-step formats, or formats having more than two incubation steps, are called “sequential” formats.

Encompassed by the present invention are homogenous, sequential and semi-sequential immunoassays as further described herein.

In a homogenous assay a capture reagent (1), a biological sample and a detection reagent (2) is premixed, the capture reagent (1) and the detection reagent (2) have equimolar concentrations, while the concentration of the analyte can be varied; thereafter the capture reagent (1), analyte, and detection reagent (2) forms a complex; which is captured on a substrate.

In a semi-sequential assay a capture reagent (1) is added to a solid phase; and the capture reagent (1) binds to the solid phase and is thereby immobilized thereon; and a biological sample is mixed with a detection reagent (2); and the analyte in the biological sample binds to the detection reagent (2) to form a complex; and the complex is added to the capture reagent (2) immobilized on the solid phase so that the complex binds to the capture reagent (1) immobilized on the solid phase.

In a sequential assay a solid phase is used to bind a capture reagent (1) added thereto, and in the next step an analyte is added, and the analyte, e.g. the ADA; binds to the capture reagent (1) immobilized on the solid phase, and in a further step a detection reagent (2) is added, and the detection reagent (2) binds to the analyte that has previously been bound to the capture reagent (1) immobilized on the solid phase.

A bridging immunoassay, as used in a method herein, involves at least three interacting molecules: A capture reagent, an analyte, and a detection reagent. In bridging immunoassays, the analyte molecule binds to both the capture reagent, and to the detection reagent, to form a complex of the type capture reagent-analyte-detection reagent, thus, the analyte forms a bridge between the capture reagent and the detection reagent utilizing the bivalency of the ADA molecule. This is also herein referred to as a bridging complex.

Accordingly, there is also provided herein an in vitro method as defined herein wherein the method comprises the steps of: a) simultaneously contacting the biological sample with said capture reagent (1) and said detection reagent (2) to obtain a reaction mixture, b) adding the reaction mixture of step a) to a solid phase allowing any bridging complex formed in step a) to attach thereto, and thereafter c) detecting any ADA from said biological sample.

Further, there is also provided herein an in vitro method as defined herein wherein the method comprises the steps of: a) adding the capture reagent (1) to the solid phase allowing the capture reagent (1) to attach thereto, b) mixing the detection reagent (2) and the biological sample to obtain a reaction mixture, c) adding the reaction mixture obtained in step b) to the solid phase of step a) allowing any ADA present in said reaction mixture to attach to the solid phase, and thereafter d) detecting any ADA from said biological sample.

There is also further provided, an in vitro method as defined herein, wherein the method comprises the steps of: a) adding the capture reagent (1) to the solid phase allowing the capture reagent (1) to attach thereto, b) adding a biological sample to the solid phase to attach any ADA present therein to said capture reagent (1), c) adding a detection reagent (2) to the solid phase treated with said biological sample, and thereafter d) detecting any ADA from said biological sample.

Further examples of assay formats that may be used are provided e.g. in WO2010042031 (Gyros patent AB).

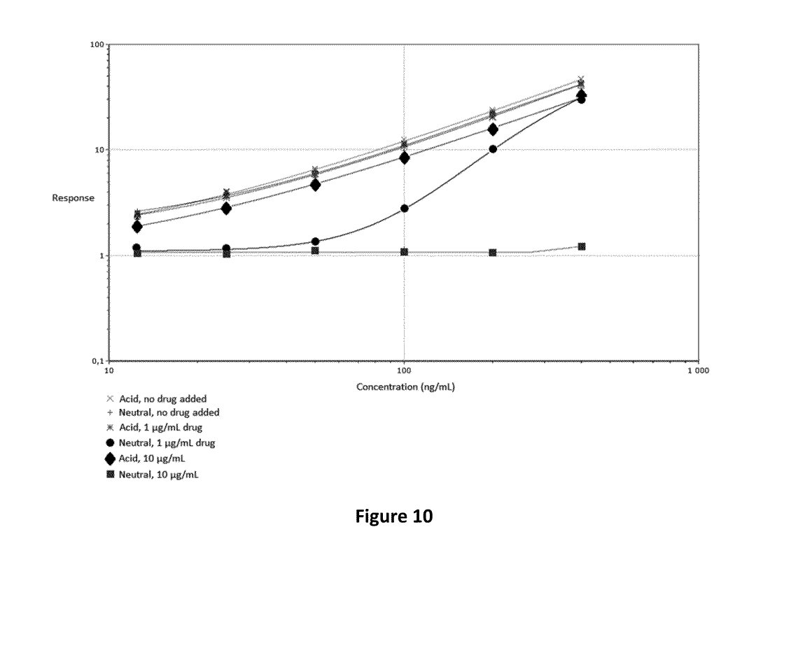

As mentioned herein, measuring the incidence of ADA using a bridging ADA assay is often interfered by therapeutic drugs remaining in patients circulation (Shankar et al. 2008). ADA molecules are complexed by the drug and can therefore not be detected by the assay. Evaluating ADA assays drug tolerance is therefore important. One common practice to avoid these problems is to dissociate drug-ADA complexes using acidic buffers even if acid may inactivate some ADAs (Li et al. 2011). Results of an acid dissociation procedure using a Fab/Fab assay are shown in FIG. 10. Therein, it was shown that Response dynamics is affected by presumed ADA-drug complexes at concentrations as low as 1 μg/mL when acidic dissociation is not used indicating the importance of acidic dissociation to improve sensitivity.

Accordingly, there is provided herein an in vitro method wherein step a) of said method is preceded by a step of adding to and incubating said biological sample with an acidic buffer before the biological sample is contacted with the capture reagent (1) and the detection reagent (2). Before the biological sample is further used in an in vitro method herein, the sample is neutralized in any suitable manner.

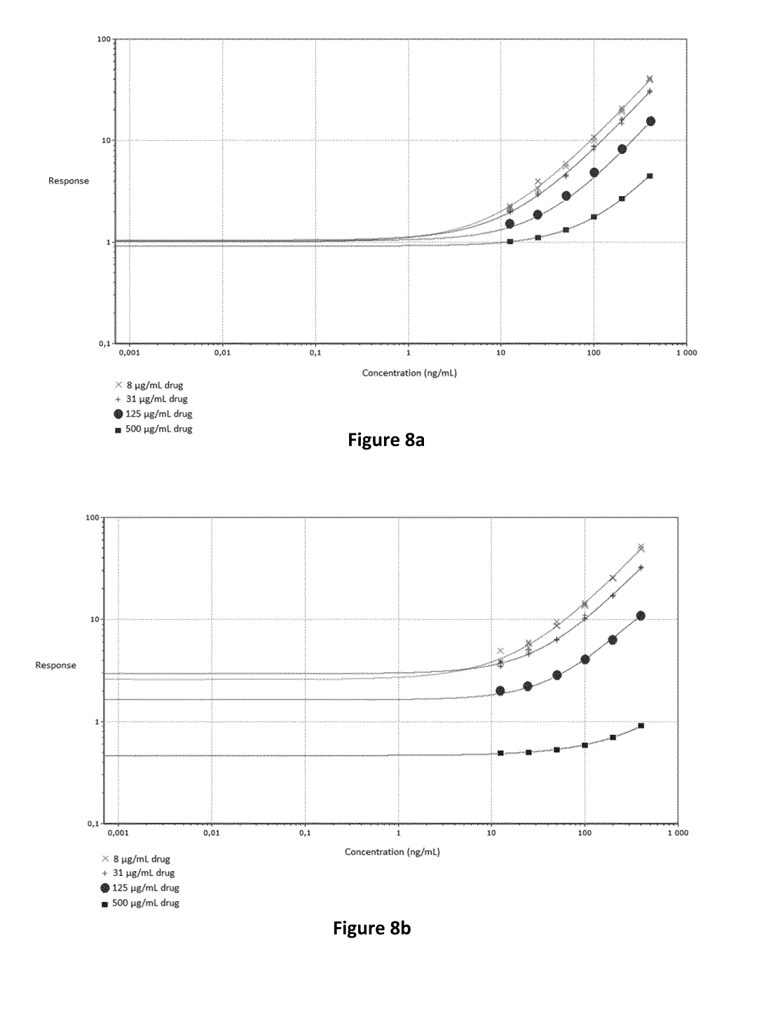

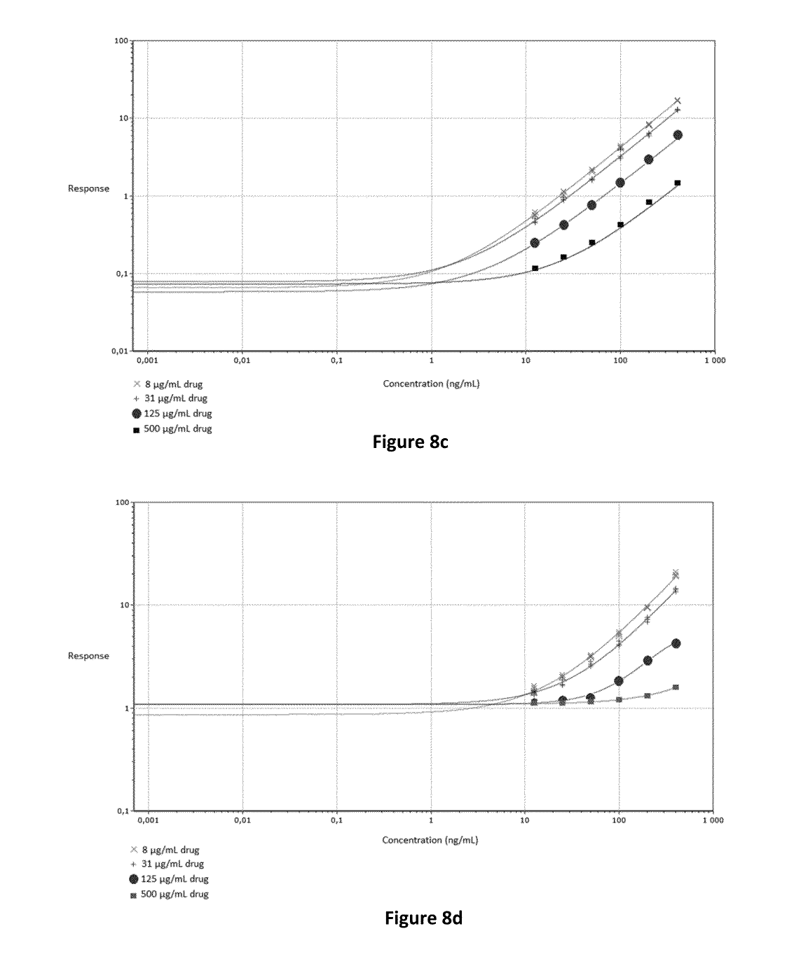

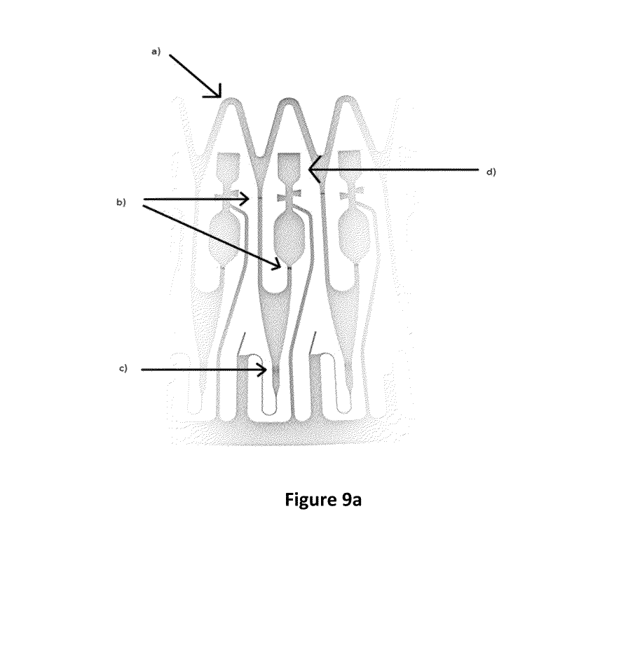

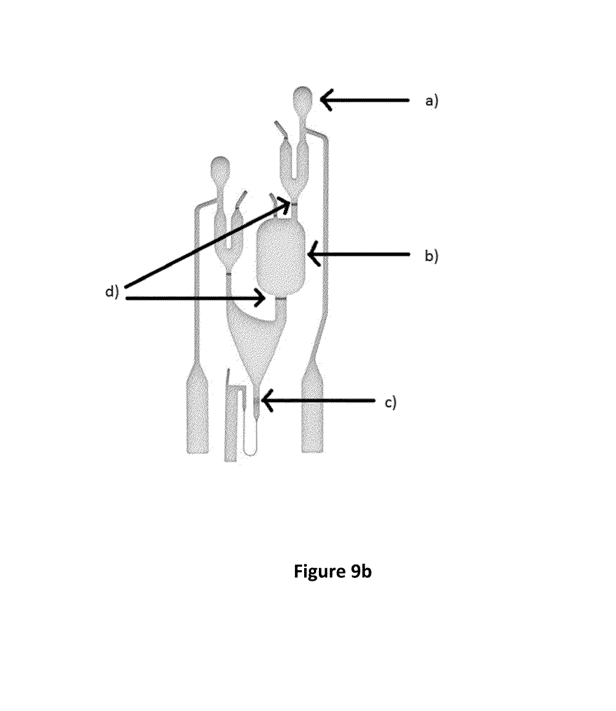

In FIG. 8a-d the results of an investigation of drug tolerance of assay variants by drug concentrations 8-500 μg/mL are shown which indicate that assay variant Intact/Fab (b-IFX and f-Fab) is the most drug tolerant assay variant.

There is a wide variety of therapeutic drug antibodies out on the market today, of which some may induce adverse drug reactions and therefore need further investigation. There is also a great need for evaluating therapeutic drug antibodies that are in clinical development to at an early stage determine if negative reactions occur, as well as decide if a patient treated routinely with a therapeutic drug antibody suffers from any negative immunological reactions during treatment.

In the table below (Table 1) are examples of therapeutic drug antibodies that may be useful in an in vitro method according to the invention.

TABLE 1
Therapeutic drug antibodies

| | | | H/M |
|---|---|---|---|
| Drug | Substance | Target | composition |
| Actemra ® | Tocilizumab | IL-6 R | IgG1k |
| Arzerra ® | Ofatumumab | CD20 | IgG1k |
| Avastin ® | Bevacizumab | VEGF | Humanized |
| | | | IgG1k |
| Benlysta ® | Belimumab | BLyS | IgG1l |
| Erbitux ® | Cetuximab | EGF-R | IgG1k |
| Herceptin ® | Trastuzumab | Her-2 | IgG1k |
| Humira ® | Adalimumab | TNFa | IgG1k |
| Ilaris ® | Canakinumab | IL-1b | IgG1k |
| MabCampath ® | Alemtuzumab | CD52 | Humanized |
| | | | IgG1k |
| MabThera ® | Rituximab | CD20 | Murine/Human |
| | | | IgG1k |
| Orthoclone ® OKT3 | Muromonab | CD3 | Mouse IgG |
| Perjeta ® | Pertuzumab | Her-2 | IgG1k |
| Prolia ® | Denosumab | RANKL | IgG2k |
| Raptiva ® | Efalizumab | CD11a of LFA1 | IgG1k |
| Remicade ® | Infliximab | TNFa | Murine/Human |
| | | | IgG1k |
| Simponi ® | Golimumab | TNFa | IgG1k |
| Simulect ® | Basilimiximab | CD25 | Murine/Human |
| | | | IgG1k |
| Soliris ® | Eculizumab | C5 | Humanized |
| | | | IgG2/4k |
| Stelara ® | Ustekinumab | IL-12/IL-23 | IgG1k |
| Synagis ® | Palivizumab | RSV | IgG1k |
| Tysabri ® | Natalizumab | a4b1 integrin | Humanized |
| | | | IgG4k |
| Vectibix ® | Panitumumab | EGFR | IgG2k |
| Xolair ® | Omalizumab | IgE | Humanized |
| | | | IgG1k |
| Yervoy ® | Ipilimumab | CD152 | IgG1k |
| | | (CTLA-4) | |
| Zenapax ® | Daclizumab | CD25 | Humanized |
| | | | IgG1 |

The in vitro method according to the invention is hence applicable to detect a wide variety of therapeutic drug antibodies having different targets and being of different origin as exemplified in the table above.

Examples of therapeutic drug antibodies are antibodies having affinity e.g. for the targets, TNF (Tumor Necrosis Factor) or TNF-α, CD20, VEGF (Vascular Endothelial Growth Factor), EGFR (Endothelial Growth Factor Receptor), CD2 and Her-2. The therapeutic antibody may be an antibody having affinity for TNF, such as TNF-α. The therapeutic drug antibody may be a humanized, chimeric or a murine antibody.

The therapeutic drug antibody (TDA) in a method herein is an antibody which provides for cleavage in the hinge region of the antibody into an Fc (fragment crystallizable) region and a F(ab′)₂ region. The thereafter obtained one or more Fab fragments, which may also be one or more monovalent Fab(s), may be used as an affinity moiety (1a, 2a) of a capture reagent (1) or a detection reagent (2) as presented herein.

Hence, there is provided an in vitro method as defined herein wherein said TDA is an intact antibody providing for cleavage thereof thereby generating one or more fab Fragment(s) from the intact antibody.

There is further provided an in vitro method wherein said TDA is selected from the group consisting of: Infliximab, Adalimumab, Bevacizumab and Denosumab. In the present in vitro method, the TDA may also be Inflimixab (Remicade®).

In the in vitro method presented herein, the capturing moiety (1b) of said capture reagent (1) may be biotin and the solid phase may comprise streptavidin for attachment of the biotin molecule thereto. Hence, this provides an example of a solid phase system useful for the present method. The biotin molecule can be attached to the affinity moiety (1b) of the capture reagent through procedures well-known in the art, including as exemplified in the experimental section. The biotin molecule of the capture reagent covalently binds to the column, or the solid support, via the streptavidin reactive sites having binding preference for the biotin molecule.

In the in vitro method herein, the detection moiety (2b) of the detection reagent (2) may comprise a luminescent label selected from the group consisting of a fluorescent label, a phosphorescent label, and a radioluminescent label. Further examples thereof are any Alexa-labels.

Further examples of labels useful in a method herein may be selected from the group consisting of: Alexa Fluor 633, Alexa Fluor 647, Dylight 649, DY-649P1, Dylight 650, Cy-5, Cy-5.5, CF 647 Dye, and Innova.

Further examples of labels for use in a method herein are provided in Mikulskis et al, 2011.

Labels may be obtained e.g. from Biotium Inc. (e.g. CF 647 Dye), Innova Biosciences, Cyanine Technologies, Seta Biomedicals, Molecular Targeting Technologies Inc., Dyomics GmbH (DY), Pierce (e.g. Dylight 649 and Dylight 650), Sigma, Dyomics GmbH (e.g. DY-649P1), Invitrogen (Alexa Fluor) and GE Healthcare (e.g. Cy-5, Cy-5.5).

The biological sample analyzed for the presence of ADA molecules in an in vitro method herein may comprise whole blood, plasma, serum, tissue homogenate or any other biological specimen from a mammal, such as a human, suitable in a method presented herein.

There is further provided herein an in vitro method wherein the method is performed using a device comprising: a biological sample inlet; a detection reagent inlet; a capture reagent inlet; and a mixing chamber in fluid contact with the sample inlet and the detection reagent inlet, wherein the mixing chamber is positioned in a direction downstream of the sample inlet and the detection reagent inlet; and a column comprising the solid support, wherein the column is in fluid contact with the biological sample inlet, the detection reagent inlet, the capture reagent inlet, and the mixing chamber, and wherein the column is positioned in a direction downstream of the capture reagent inlet and the mixing chamber; and an outlet in fluid contact with the column, wherein the outlet is positioned in a direction downstream of the column; and a means for detecting the detection reagent.

WO 2005/094976 A1 (Gyros patent AB) discloses a mixing unit which is useful for performing the in vitro method according to the invention. Other examples of microfluidics applicable to the present invention are WO 03/018198 A1 (Gyros patent AB) and WO 2008/103116 (Gyros patent AB).

Other examples of technology platforms for performing an in vitro method as presented herein are e.g. solid phase ELISA (Enzyme-linked Immunosorbent Assay) technology platforms, the AlphaLlSA platform and ECL (Electrochemiluminescent) platforms, and as exemplified in Mikulskis et al, 2011.

The in vitro method presented herein, may be performed by using immunoassays including a microfluidic technique optionally in a spinning compact disc (CD). The method is then in a performed in a nanoliter-scale.

Accordingly, the in vitro method disclosed herein may be performed on Gyrolab™ (Gyros AB), as further described herein (Barry & Ivanov 2004), but is not limited thereto. The in vitro method may be performed in an array format.

The Gyrolab™ workstation enables efficient development of nanoliter-scaled immunoassays by using a microfluidic technique in a spinning compact disc (CD) (Barry & Ivanov 2004). The workstation is computer controlled and transfer liquids from a microtiter plate onto a CD by fully automated methods (Dudal et al. 2014). The use of a CD microlaboratory enables small sample volumes (<10 μl) to be used (Joyce et al. 2014). In order to increase assay performance in Gyrolab assay buffers called Rexxip™ are used (Gyros AB 2014a).

The CD (Gyrolab CD microlaboratory) contains a number of microstructures where samples are added (Dudal et al. 2014). Inside each microstructure there is a capture column pre-packed with streptavidin-coated beads (Liu et al. 2012). One microstructure is therefore needed for each data point (Gyros AB 2011). Streptavidin is a tetrameric bacterial protein with extremely high affinity for biotin (Janeway et al. 2001). The Gyrolab™ workstation utilizes biotinylated (b) capture reagents (Liu et al. 2012).

There are two types of Gyrolab CD microlaboratories, Gyrolab Bioaffy™ CD and Gyrolab Mixing™ CD. There are three types of Gyrolab Bioaffy™ CDs, Bioaffy 1000, Bioaffy 200 and Bioaffy 20HC. The major difference between these microlaboratories is the sample volume added to the column, which are 1000 nL, 200 nL and 20 nL respectively (Gyros AB 2011).

Gyrolab Bioaffy™ CDs contain different functional units. In the common channel the b-capture reagent, f-detection reagent and wash solutions are added and distributed by capillary force. In the individual inlet the analyte is added and distributed by capillary force. Hydrophobic barriers stop solutions from flowing in an uncontrolled manner in the CD. Centrifugal force is used to move liquids within the CD in a controlled manner by spinning the CD. At the same time the centrifugal force overcome the hydrophobic barriers and liquids are eventually transferred to the capture column (Gyros AB 2014b). Gyrolab methods are programmed to add capture reagent, detection reagent, wash solutions and analyte solutions in desired order (Gyros AB 2011).

Gyrolab Mixing™ CD enables an automated sample pretreatment before samples are added to the capture column.

A key application for this microlaboratory is ADA analysis where samples can be treated with acid to dissociate proteins in complex before being added to the column (Gyros AB 2014c). The Gyrolab Mixing™ CD contains different functional units. In the inlet, sample containing analyte is added and distributed by capillary force. A hydrophobic barrier stop sample from flowing in an uncontrolled manner in the CD and sample volume is defined to 200 nL. Next centrifugal force is applied by spinning the CD to overcome the hydrophobic barrier and sample flows into the mixing chamber where sample flow is stopped by another hydrophobic barrier. In the next step acidic buffer is added and mixed with the sample to dissociate any preformed ADA-Drug complexes. Finally the pH of the mixture is elevated to neutral by addition of detecting reagent in neutral buffer, allowed to mix for a preset time to allow complex formation of ADA and assay reagents. By spinning the CD the complex is added to the streptavidin column (Gyros AB 2014c). Gyrolab methods are programmed to add capture reagent, detection reagent, wash solutions and analyte solutions in desired order (Gyros AB 2011).

The Gyrolab™ workstation utilizes fluorescently (f) labeled detecting molecules (Liu et al. 2012). Once the reaction is completed the fluorescent signal is detected with laser induced fluorescence on the surface of the capture column (Inganas et al. 2005). When the signal (emitted light from the fluorophore) reaches the detector it is amplified using a photo multiplier tube (PMT). The degree of amplification that is needed for detection depends on signal intensity from the measured sample. Amplification levels are controlled by Gyrolab methods and are expressed as percentage of incoming signal (Gyros AB 2011).

It is a further objective of the present invention to provide the use of an in vitro method as presented herein for the detection of an anti-drug antibody (ADA) against a therapeutic drug antibody (TDA) in a biological sample, for determining if a patient or subject treated with an therapeutic drug antibody has developed a sensitivity or an adverse drug reaction against said therapeutic drug antibody.

Accordingly, there is provided herein an in vitro method for identifying if a patient treated with a therapeutic drug antibody (TDA) has developed ADA's against a therapeutic drug antibody and/or is at risk at developing a sensitivity, adverse drug reaction or develop negative immune reactions against said TDA comprising performing an analysis of a biological sample as described by a method herein.

There is further provided herein a kit comprising means for performing the in vitro method presented herein, i.e. said kit comprising a detection reagent, a capture reagent of the type mentioned herein as well as other reagents suitable for performing the method.

Accordingly there is provided a kit for performing an in vitro method as defined herein, wherein said kit comprises: a) a capture reagent (1) having an affinity moiety which comprises an intact TDA and a detection reagent (2) having an affinity moiety (2a) which comprises one or more Fab fragment(s) of said TDA, or b) a capture reagent (1) having an affinity moiety (1b) which comprises one or more Fab fragment(s) of a TDA and a detection reagent (2) comprising an intact TDA, and optionally instructions for use.

Optionally instructions for use are also provided with said kit. The kit may be complemented with any of the reagents or means described herein.

The invention is hereafter illustrated in the following experimental section, however the invention is not limited thereto the scope instead being determined by the appended set of claims.

### EXPERIMENTAL SECTION

### Abbreviations

Abs: Antibodies
ADA: Anti-drug antibody
b: Biotinylated
β-ME: β-mercaptoethanol
BSA: Bovine serum albumin
CD: Compact disc
CF: Correction factor
CP: Cut point
CV: Coefficient of variation
DOL: Degree of labeling
DTT: Dithiothreitol
f: Fluorescently labeled
Fab: Fragment antigen binding
Fc: Fragment crystallizable
FCP: Floating cut point
h: hours
IFX: Infliximab
Ig: Immunoglobulin
kDa: Kilodalton
mAbs: Monoclonal antibodies
MW: Molecular weight
PBS: 15 mM phosphate buffer, 150 mM NaCl, pH 7.4
PMT: Photo multiplier tube
RA: Rheumatoid arthritis
RF: Rheumatoid factor
S/B: Signal to background
SDS-PAGE: Sodium dodecyl sulfate polyacrylamide gel electrophoresis TNF: Tumor necrosis factor

### Experimental

All experiments included herein are in vitro experiments and human samples are handled in a laboratory used for analysis of human samples. Anti-IFX used in this project is commercially bought according to valid laws and regulations. IFX used in this project is prescribed by a physician and bought according to valid laws and regulations. Results from this project are not used to evaluate patient treatments and patients distributing samples remain anonymous.

### Material

### Consumables

FragIT™ MidiSpin Kit (A2-FR2-100) was purchased from Genovis (Lund, Sweden). Amicon® Ultracel 30K centrifugal filter (UFC803024) and Amicon® Pro 30K centrifugal filter (ACS503012) were obtained from Merck Millipore (Solna, Sweden). Protein Desalting Spin Columns (89849) and Zeba™ Spin Desalting Columns 0.5 mL (89882) were obtained from Thermo Scientific (Stockholm, Sweden).

CH₃COOH (1.00063.1000), CH₃OH (1.06009.1000) and NaOH (1.06498.1000) were obtained from Merck Millipore. DL-Dithiothreitol (D9779-IG), Glycerol (G-5516) and 0.22 μM Membrane Filter (60301) were obtained from Sigma Life Science (Stockholm, Sweden). Sodium dodecyl sulfate (00307) was obtained from VWR (Stockholm, Sweden). PhastGel™ Gradient 8-25 (17-0542-01), PhastGel™ Sample applicator 6/4 (18-0012-29), PhastGel™ SDS Buffer Strips (17-0516-01), PhastGel™ Blue R (17-0518-01) and Amersham™ LMW Calibration Kit For SDS Electrophoresis (17-0446-01) were obtained from GE Healthcare (Uppsala, Sweden). 96-well PCR Plates (AB-0800) were obtained from Thermo Scientific. Alcojet® Detergent powder was obtained from Alconox (White Plains, USA).

Gyrolab™ Mixing CD (P0020026) and Gyrolab Bioaffy™ 200 (P0004180) were obtained from Gyros (Uppsala, Sweden).

### Reagents

Infliximab (trade name Remicade) was obtained from Apoteket Akademiska (Uppsala, Sweden). Anti-idiotype IgG1 directed against IFX (clone 17841-hIgG1) were obtained from AbD Serotec (Oxford, England).

EZ-Link® Maleimide-PEG2-Biotin (21902) and EZ-Link® Sulfo-NHS-LC-Biotin (21327) were purchased from Thermo Scientific. Alexa Fluor® 647 Antibody Labeling Kit (A20186) and Alexa Fluor® 647 C2-maleimide (A20347) were purchased from Invitrogen (Udine), Sweden). Bovine serum albumin 10% (126615) was obtained from Merck Millipore, former Calbiochem (San Diego, USA). Rexxip™ ADA (P0020027) was obtained from Gyros. Glycine (1.04201.1000), HCl (1.00316.100), NaCl (1.06404.1000), NaH₂PO₄ (1.06346.1000), Na₂HPO₄ (1.06586.0500), Tris (1.08219.1000) and Tween® 80 (8.22184.0500) were obtained from Merck Millipore.

### Samples

Human serum pool (S-123-V) and individual human serum (SIM-123-V and SIF-123-V) were obtained from Seralab (Haywards Heath, England). IFX patient samples were provided by the Department of Medical Sciences at Uppsala University. Positive RF patient samples were provided by the department of Immunology, Genetics and Pathology at Uppsala University and the Department of Clinical and Experimental Medicine Rheumatology at Linkoping University.

### Systems

Nanophotometer (6133) was supplied by LabVision (Stockholm, Sweden). Gyrolab™ workstation was supplied by Gyros. PhastSystem™ (18-1018-24) was supplied by GE Healthcare, former Pharmacia Biotech (Uppsala, Sweden).

### Extinction Coefficients for Determination of Protein Concentration

To determine protein concentration for F(ab′)2 fragments and b-molecules absorbance was measured at 280 nm. Protein concentration was calculated according to Equation 1. The extinction coefficient has a specific value for each molecule, these are listed in Table 2.

Equation 1. Calculation of protein concentration for F(ab′)2 fragments and biotinylated molecules.

$$Protein concentration ( mg \text{/} mL ) = \frac{{A}_{280} \cdot dilution factor}{extinction coefficient \cdot cuvette length}$$

TABLE 2
Extinction coefficients for Immunoglobulin G, F(ab′)₂ and Fab fragments.

| Molecule | Extinction coefficient [cm⁻¹(mg/mL)⁻¹] |
|---|---|
| Fab | 1.53^{b} |
| F(ab′)₂ | 1.48^{b} |
| IgG | 1.38^{c} |

^{b}Andrew & Titus 2000
^{c}Gyros AB 2011

### Protein Concentration and Degree of Labeling for Fluorophore Labeled Molecules

The procedure was performed according to manufacturer's instructions; To determine protein concentration for f-molecules absorbance was measured at 280 nm and 650 nm. Protein concentration was calculated according to Equation 2 and degree of labeling (DOL) was calculated according to Equation 3. Extinction coefficients are listed in Table 2.

Equation 2. Calculation of protein concentration for fluorophore labeled molecules.

$$Protein concentration ( mg \text{/} mL ) = \frac{( {A}_{280} - ( {A}_{650} \cdot 0.03 ) ) \cdot dilution factor}{extinction coefficient}$$

Equation 3. Calculation of degree of labeling for fluorophore labeled molecules.

$$Degree of labeling = \frac{{A}_{650} \cdot dilution factor}{extinction coefficient \cdot protein concentration ( mg \text{/} mL )}$$

Digestion of IFX into F(Ab′)2 Fragments

Digestion of IFX and purification of IFX F(ab′)₂ fragments were performed using a kit from Genovis (FragIT kit A2-FR2-100). The kit consists of two spin columns, FragIT column and CaptureSelect column. The FragIT column containing an immobilized enzyme (FabRICATOR) cleaves the mAbs in one specific site below the hinge region leaving a F(ab′)₂ fragment and a Fc fragment. The CaptureSelect column contains immobilized antibody fragments directed towards the Fc portion of IgG, effectively separating the Fc fragments from F(ab′)₂ fragments.

### Cleavage of IFX and Isolation of F(Ab′)2 Fragments

The following steps were done according to manufactures instructions using protocol FragIT™ MidiSpin Kit (A2-FR-100). FragIT spin column was centrifuged at 100×g for 1 minute. The column was equilibrated using 2.5 mL cleavage buffer (50 mM sodium phosphate, 150 mM NaCl, pH 6.6) and centrifuged at 100×g for 1 minute. The equilibration step was performed three times. 10 mg IFX diluted in cleavage buffer to a final concentration of 5 mg/mL was added to FragIT spin column and the mix was allowed to incubate at room temperature by end-over-end mixing for 30 min. The sample was eluted by centrifugation at 100×g for 1 minute. For maximal sample recovery 1 mL cleavage buffer was added and the column was centrifuged at 100×g for 1 minute, this step was performed twice.

CaptureSelect spin column was centrifuged at 200×g for 1 minute. The column was equilibrated using 3 mL binding buffer (10 mM sodium phosphate, 150 mM NaCl, pH 7.4) and centrifuged at 200×g for 1 minute. The equilibration step was performed three times. The eluted sample from the cleavage step was added and the mix was allowed to incubate at room temperature by end-over-end mixing for 30 min. The sample was eluted by centrifugation at 200×g for 1 minute. For maximal sample recovery 1 mL binding buffer was added and the column was centrifuged at 200×g for 1 minute, this step was performed twice but during the last centrifugation the column was centrifuged at 600×g for 1 minute.

After separation a protein concentration was performed using an Amicon® Ultracel 30K centrifugal filter. The following steps were done according to manufactures instructions using protocol Amicon® Ultra-4 Centrifugal Filter Devices. The sample were added to Amicon® Ultracel 30K spin column and centrifuged at 4000×g for 10 minutes. The sample was recovered using a pipette.

Protein concentration was determined after absorbance measurement (Equation 1).

Evaluation of Digestion Procedures Using SDS-PAGE with Coomassie Staining

To evaluate the digestion process SDS-PAGE with Coomassie staining was performed. The following steps were done according to Protocol Separation Technique File No. 110 Phastsystem (GE Life Sciences). Samples were diluted into 0.3-1 mg/mL and treated with sample solution 1 (2.5% sodium dodecyl sulfate and 5% β-ME) and 1% bromophenol blue. For comparison samples were also treated with sample solution without 5% β-ME. Samples were heated at 100° C. for 5 minutes, centrifuged at 13000 rpm for 2 minutes and loaded on PhastGel 8-25 using a sample applicator 6/4. Gels were placed in the separation compartment and SDS buffer strips were placed in the buffer strip holder.

After separation gels were transferred to the development chamber. Gels were dyed with Coomassie staining solution (0.1% PhastGel Blue R, 30% methanol and 10% acetic acid). Gels were destained using destaining solution (30% methanol and 10% acetic acid) and finally treated with storage solution (13% glycerol and 10% acetic acid). Gels were allowed to dry over-night.

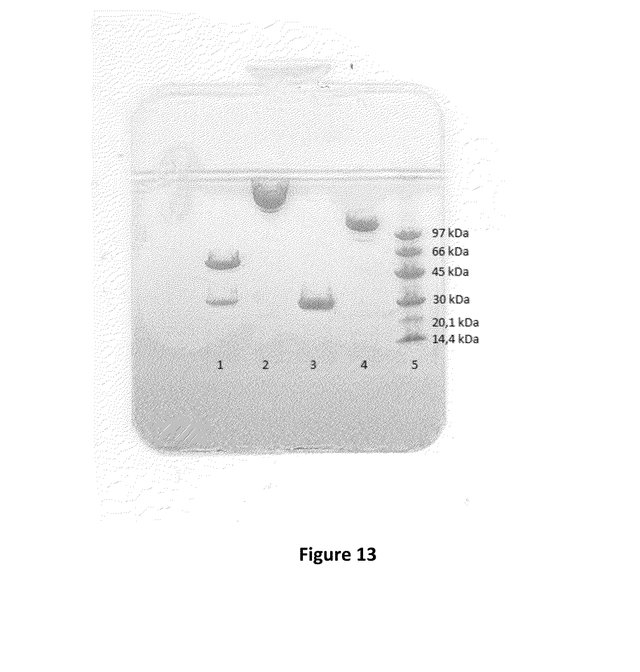

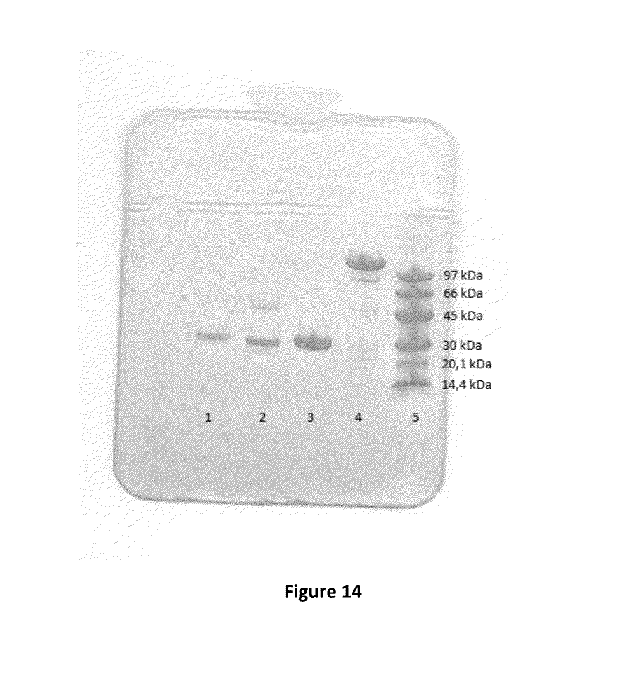

As reference a sample containing proteins with MW 14.4-97 kDa was used and treated like the rest of the samples. Results are shown in FIGS. 13 and 14.

### Labeling Procedures

### Desalting

### Amicon® Pro 30K Centrifugal Filters

For separating DTT and unreacted Alexa 647-C2-maleimide from proteins Amicon® Pro 30K centrifugal filters were used. Amicon® Ultra 0.5 mL filter and exchange device were placed in 50 mL collection tube. Sample and 300 μl binding buffer were added to the exchange device followed by centrifugation at 4000×g for 4 minutes. 2.5 mL binding buffer was added to the exchange device followed by centrifugation at 4000×g for 12 minutes. To recover sample the filter was placed upside down in 0.5 mL filter collection tube and centrifuged for 1000×g for 2 minutes.

### Desalting Spin Column

For separating unreacted biotin reagent from proteins desalting spin columns were used. The following steps were done according to manufactures instructions using protocol Instruction protein desalting spin columns (89862). The spin column was centrifuged at 1500×g for 1 minute. Sample (30-120 μl) was added to the spin columns resin. The spin column was centrifuged at 1500×g for 2 minutes.

### Reduction of F(Ab′)2 Fragments

F(ab′)2 fragments were reduced using 2 mM DTT (Kan et al. 2001). The mixture was allowed to incubate for 60 minutes (Lee et al. 2005) in room temperature with gentle mixing and separated on Amicon® Pro 30K centrifugal filters.

### Biotinylation

### Biotinylation of IFX

The following steps were done according to protocol B1.1.

Biotinylation of IFX was done according to manufacturer's instructions using protocol EZ-Link® Sulfo-NHS-LC-Biotin (21327) (Gyrolab user guide D0016423). IFX was diluted to a final concentration of 1 mg/mL and mixed at 12 times molar excess of biotinylation reagent. The mixture was allowed to incubate 1 hour at room temperature with some mixing and separated on a desalting spin column.

Protein concentration was determined after absorbance measurement (Equation 1). The solution was stored in 4-8° C.

### Biotinylation of IFX Fab Fragments

Before biotinylation of Fab fragments, IFX F(ab′)₂ fragments were reduced. The following steps were done according to manufactures instructions using protocol EZ-Link® Maleimide-PEG₂-Biotin (21902). Biotin reagent was diluted to a final concentration of 20 mM. 25 times molar excess of biotin reagent was chosen and mixed with Fab fragments. The mixture was allowed to incubate at +4° C. over-night and separated on a desalting spin column. Protein concentration was determined after absorbance measurement (Equation 1). The solution was stored in 4-8° C.

### Fluorophore Labeling

### Fluorophore Labeling of IFX

Fluorophore labeling of IFX was performed using a kit from Life Technologies (manufacturer's instructions using protocol Alexa Fluor® 647 Monoclonal Antibody Labelling Kit (A20186)). The following steps were done according to protocol B1.2 Fluorophore labeling of detection reagent in Gyrolab user guide D0016423. IFX was diluted to a final concentration of 1 mg/mL and a tenth volume of 1 M Sodium bicarbonate buffer were added. The mixture was transferred to the vial containing the active dye, the vial was wrapped in aluminum foil. The mixture was allowed to incubate for 1 hour at room temperature with some mixing. A desalting column from the kit was used for separation. The column was packed with the purification resin from the kit to a final bed volume of approximately 1.5 mL. The mixture was added to the purification resin and the column was centrifuged at 1100×g for 5 minutes.

Protein concentration and DOL were determined after absorbance measurement (Equation 2 and 3). The labeled protein was diluted to 1 μM in 15 mM phosphate buffer, 150 mM NaCl, pH 7.4 (PBS) and 0.2% Bovine serum albumin (BSA) and stored in dark vials in −20° C.

### Fluorophore Labeling of IFX Fab Fragments

Before fluorophore labeling of Fab fragments, IFX F(ab′)₂ fragments were reduced. The following steps were done according to manufactures instructions using protocol Thiol-reactive probes (A20347). The reactive dye was diluted to a final concentration of 10 mM in binding buffer. 20 times molar excess of reactive dye was chosen and mixed with Fab fragments. The vial was wrapped in aluminum foil and the mixture was allowed to incubate in +4° C. over-night. The mixture was separated on Amicon pro 30K centrifugal filter. Protein concentration and DOL were determined after absorbance measurement (Equation 2 and 3). The labeled protein was diluted to 2 μM in binding buffer, 0.2% BSA and stored in dark vials in −20° C.

### Immunoassay Analysis Using Gyrolab™ Workstation

### Gyrolab Mixing™ CD

On the mixing CD both standard series of analyte and unknown samples were analysed. Standard series corresponding to 125-4000 ng/mL anti-IFX and diluted in neat serum or Rexxip™ ADA were used. When drug tolerance was analysed standard series were diluted in neat serum with 8-500 μg/mL IFX and allowed to incubate for 1 h at room temperature in tubes (no shaking of tubes during incubation). Neat serum or Rexxip™ ADA was used as blank samples. When unknown samples were analysed neat pool serum was used as negative controls and 250-500 ng/mL anti-IFX was used as positive controls.

Standard series, blank samples, unknown samples, positive controls and negative controls were diluted 1:10 using Rexxip™ ADA before analysis on mixing CD.

0.5 M Glycine-HCl pH 2.6 was used as acidic buffer and capture and detection reagents were diluted in equal parts of 2 M Tris-HCl pH 8.0 and Rexxip™ ADA. When the effects of acid treatment were evaluated Rexxip™ ADA was exchanged for acidic buffer.

### Bioaffy 200 CD

On Bioaffy 200 CD standard series were analysed. Standard series corresponding to 125-4000 ng/mL anti-IFX diluted in neat serum or Rexxip™ ADA were used. Neat serum or Rexxip™ ADA was used as blank samples. Samples and blank were diluted 1:10 using Rexxip™ ADA. Capture and detecting reagents were mixed and then mixed manually with samples (10 μL+10 μL). Mixtures were incubated for 1 h in room temperature before analysis in Gyrolab™.

### Designing and Executing Gyrolab Runs

Each Gyrolab run was executed according to Gyrolab User Guide (version P0004354, 2011) and Gyrolab ADA assay protocol (version D0016561, 2014) using the software Gyrolab Control. Runs were designed using preexisting Gyrolab methods Bioaffy 200 1-step wash×2 wiz and ADA-1W-003-A. Execution of runs contained information about CD consumption and generated the document Gyrolab Control Loading List. Gyrolab Control Loading List contains name, concentration, volume and micro plate and well position for all sample types. Sample types included in this project were capture and detection reagents, standard series, unknown samples, wash buffer and acid solutions. Samples were prepared differently depending on CD type and added to micro titer plates according to Gyrolab Control Loading List. Micro titer plates were sealed with foil and centrifuged at 3000×g for 2 minutes. The Gyrolab was loaded with CDs and micro titer plates, wash station 1 and pump station 1-5 were connected to PBS, 0.02% NaN₃, 0.01% Tween 20 (0.22 μM syringe filtered), wash station 2 was connected to wash solution 2 and the run was executed. After a finished run CDs and micro titer plates were removed from Gyrolab and data was analysed according to a five parameter logistic model with weight response (standard series) and weight concentration (unknown samples) on software Gyrolab Evaluator (version 3.3.7.171), PMT 5% was used.

### Statistical Tools

### Shapiro-Wilk Normality Test

To determine CP the data set should be normally distributed and one method to decide if a data set is normally distributed is Shapiro-Wilk Normality Test (Shankar et al. 2008). The statistical calculations used are described by Shapiro & Wilk (1965). To calculate if the data set was normally distributed an online calculator was used in this project (Shapiro-Wilk normality test 2014).

### Grubbs' Test

To determine CP significant outliers in the data set should be identified and excluded (Shankar et al. 2008). A response value is said to be a significant outlier if it deviates markedly from the rest of the dataset (Grubbs 1969). One method to identify significant outliers is Grubbs' test and the statistical calculations used are described by Grubbs (1969). To calculate if significant outliers were present in the data set an online calculator was used in this project (Graphpad™ software 2014).

### Cut Point Determination

Before CP determination data sets were controlled to be normally distributed and significant outliers were identified. CP is calculated by multiplying the standard deviation with 1.645 and then adding the average of measured values (Shankar et al. 2008). CP was calculated according to Equation 4.

Equation 4. Calculation of cut point.

Cut point=average signal response+(1.645·standard deviation)

To include possible differences in execution a FCP for each run is calculated. The FCP is based on the average response for negative controls in one run and a correction factor (CF) based on the calculated CP. The CF is the difference between CP and average response for negative controls from CP determination (Shankar et al. 2008). The FCP is calculated according to Equation 5 and the CF is calculated according to Equation 6.

Equation 5. Calculation of floating cut point.

Floating cut point=average response for negative controls in one run+correction factor

Equation 6. Calculation of correction factor.

Correction factor=cut point−average response for negative controls from cut point determination

### Results and Discussion

### Analysis

IFX was successfully digested and reduced into Fab fragments, which together with IFX were successfully labeled. This enabled development of ADA assay variants based on Fab fragments which could be compared to an assay based on intact reagents. These four assay variants were tested for drug tolerance and evaluated based on assay sensitivity and false negative reactions. This was done by several experiments including optimizing reagent concentration, premix testing and evaluation of blank samples. CPs for both the Fab assay and IFX assay were determined. RF samples and IFX patient samples were then finally screened

Results from Digestion and Reduction Processes Using SDS-PAGE

To evaluate digestion and reduction procedures SDS-PAGE with Coomassie staining was performed. The results from SDS-PAGE indicate that digestion and reduction of IFX was successful. Non-reduced Fab fragments are present. Fab fragments should have a MW of 50 kDa and the presence of intact Fab fragments is indicated by a lighter band. A stronger band is visible at ˜25 kDa indicating that the light chain and cleaved heavy chain are separated. Fab′ fragments probably had difficulties staying intact during SDS and heating treatment and therefore a stronger band is present at 25 kDa. Results are shown in FIGS. 13 and 14.

### Developing a Functioning ADA Assay Containing Drug Fab Fragments

When developing a functioning ADA assay containing drug Fab fragments three different assay variants were evaluated. These variants were b-Fab fragment and f-Fab fragment, b-Fab fragment and f-IFX, b-IFX and f-Fab fragment. For comparison an ADA assay containing intact reagents was also developed. A schematic figure of evaluated ADA assays is presented in FIG. 1a-d. The different assay variants were evaluated in terms of signal intensity and assay sensitivity, the level where signal response can be distinguished from background response, in several experiments.

### Different Reagent Concentrations

To investigate assay sensitivity and signal intensity, assay variant b-Fab and f-Fab (FIG. 1b) and b-IFX and f-IFX (FIG. 1d) were evaluated using four equimolar reagent concentrations. Standard curves can be found in FIG. 2a-d. As presented in FIG. 2b-c the Fab assay show higher signal response and lower background response compared to the IFX assay in all reagent concentrations. In FIG. 2a the Fab assay is presumed to show lower background response for lower standard points and in FIG. 2d the Fab assay is presumed to show higher signal response for higher standard points.

Evaluation Using Different Diluents

To investigate assay sensitivity and signal intensity blank sample and standard points of anti-IFX were diluted in neat serum or Rexxip ADA. Assay variant b-Fab and f-Fab (FIG. 1b) and b-IFX and f-IFX (FIG. 1d) were used. The standard curve is presented in FIG. 3.

As presented in FIG. 3 the Fab assay displayed lower background response than the IFX assay in Rexxip™ ADA dilution and higher background response than the IFX assay in neat serum dilution. The Fab assay displayed higher signal responses than the IFX assay in both serum and Rexxip™ ADA.

### Evaluation of Four Different Assay Variants

To investigate assay sensitivity and signal intensity all different assay variants were evaluated (FIG. 1a-d). Standard curves can be found in FIG. 4, anti-IFX concentrations displaying signal to background (S/B)>2 can be found in Table 3 and CV for response in blank samples can be found in Table 4.

As displayed in FIG. 4 assay variant b-IFX and f-Fab results in lowest background response and lowest signal response. Assay variant b-Fab and f-IFX results in highest background response and highest signal response. Table 3 indicate assay variant b-IFX and f-Fab display best response dynamics and Table 4 displays all assay variants besides b-IFX and f-Fab display CV<20% in blank samples.

TABLE 3
Anti-Infliximab concentrations displaying signal responses at least twice
as high as the background response, for the four evaluated assay variants.

| | Analyte conc. | |
|---|---|---|
| Assay variant | interval (ng/mL) | S/B interval |
| b-IFX and f-Fab | 125-4000 | 7-180 |
| b-Fab and f-Fab | 250-4000 | 2.5-26 |
| b-Fab and f-IFX | 500-4000 | 2.5-15 |
| b-IFX and f-IFX | 500-4000 | 2.5-15 |

TABLE 4
CV of response in blank samples for the four evaluated assay variants.

| | CV response |
|---|---|
| Assay variant | (%) |
| b-IFX and f-Fab | 40 |
| b-Fab and f-Fab | 5 |
| b-Fab and f-IFX | 3 |
| b-IFX and f-IFX | 5 |

### Background Experiment

To further investigate background response, samples only containing detection reagent were studied. Six samples containing 40, 120 and 360 nM f-Fab and 20, 60 and 180 nM f-IFX were mixed manually with 10% serum and analysed in 6-plicate on Bioaffy 200. Column profiles are displayed in FIGS. 11 and 12 (40 nm f-Fab and 20 nm f-IFX).

Samples containing only f-Fab result in barely detectable background response. Increasing detection concentration 3 times resulted in 2 times increase in background. Increasing detection concentration 9 times resulted in 4 times increase in background.

Samples containing only f-IFX result in detectable background response. Three times increased detection concentration results in 3 times increase in background. Increasing detection concentration 9 times results in 8 times increase in background.

### Cut Point Determination

After titration 20 nM b-IFX, 120 nM f-Fab and 20 nM f-IFX were used (data not shown). These reagent concentrations resulted in average blank response 0.1-0.2 and blank CV response<20% for both assay variants.

The CP was determined according to Equation 4 for assay variants b-IFX and f-Fab and b-IFX and f-IFX using serum samples from blood donors. 50 patient samples were screened for the presence of anti-IFX by determine a FCP (Equation 5) based on the determined CF (Equation 6). To evaluate false positive reactions 19 positive RF samples, evaluated using a nephelometer, were also screened using a FCP. As negative control neat serum was used and as positive control 250 ng/mL and 500 ng/mL anti-IFX were used. Samples, positive controls and negative controls were diluted 1:10 in Rexxip™ ADA before analysis on Gyrolab Mixing™ CD with acid dissociation.

TABLE 5
Determination of cut point

| | Biotinylated | Biotinylated |
|---|---|---|
| | Infliximab and | Infliximab and |
| | fluorophore | fluorophore |
| | labeled Fab | labeled Infliximab |
| 25 blood donor samples | 0.24 | 0.22 |
| (Cut point) | | |
| Correction factor | 0.08 | 0.08 |
| Infliximab patient samples 1-25 | 0.18 | 0.21 |
| (Floating cut point) | | |
| Infliximab patient samples 26-50 | 0.19 | 0.21 |
| (Floating cut point) | | |
| Positive Rheumatoid factor | 0.18 | 0.22 |
| samples 1-10 (Floating cut point) | | |
| Positive Rheumatoid factor | 0.15 | 0.15 |
| samples 11-19 (Floating cut point) | | |

A seen in the table above, CP was determined to 0.24 for assay variant b-IFX and f-Fab and to 0.22 for assay variant b-IFX and f-IFX.

As can be seen in FIGS. 6a and 6b no blood donor samples screened positive for b-IFX and f-Fab while 2 screened positive for b-IFX and f-IFX. Further, 27 of 50 IFX patient samples (54%) screened positive for both assay variants. Nine positive IFX patient samples (18%) indicated to be definitely positive showing average response values at least two times FCP for b-IFX and f-Fab. 14 positive IFX patient samples (28%) indicated to be definitely positive showing average response values at least two times FCP for b-IFX and f-IFX.

At last, no positive RF samples screened positive for b-IFX and f-Fab while 13 (68%) screened positive for b-IFX and f-IFX. Four of those positive samples (21%) indicated to be definitely positive showing average response values at least two times FCP.

### Evaluation of Assay Sensitivity

Different factors can be involved in assay sensitivity. Hypothetically, one of these factors is reagent aggregation, meaning that the capture and detection reagents bind each other by unspecific interactions. Hypothetically, if capture and detection reagents are complexed signal will be detected without ADA present. This detection will occur as background and be visible in blank samples. If IFX is dissolved incautiously, for example if vials are vigorously shaken, aggregation of drug molecules can take place.

In one run assay format b-Fab and f-Fab were compared to b-IFX and f-IFX when diluted in Rexxip™ ADA and neat serum (FIG. 3). Fab fragments displayed lower background signals than IFX when diluted in Rexxip™ ADA. One explanation for this could be that IFX has greater tendency for unspecific binding compared to Fab fragments, it seems when the Fc part is removed reagent molecules have a lower tendency to aggregate.

Unlike when diluted in Rexxip™ ADA, Fab fragments displayed higher background response than IFX when diluted in neat serum. These findings indicate an assay based on Fab fragments could be affected by some serum factor. One explanation for these findings could be the presence of human anti-IgG hinge autoantibodies. Anti-IgG hinge autoantibodies bind to the hinge region of a cleaved antibody but not to the intact IgG counterpart (Brerski, Knight & Jordan 2011). Hypothetically, the presence of anti-IgG hinge autoantibodies would act as a bridge between b-Fab fragments and f-Fab fragments hence cause detection and increased background.

Further evaluation of different assay formats led to the comparison between three different assay formats containing Fab fragments to an assay of intact reagents (FIG. 4). Assay format b-IFX and f-Fab indicates approximately 25-70 times lower background response compared to the other assay variants. Assay variant b-Fab and f-IFX display 70 times higher background response compared to assay variant b-IFX and f-Fab. Therefore background response cannot be solely explained by aggregate formation since these two variants reasonably should result in similar background response. One explanation for the increase seen in background could be unspecific interactions between IFX Fc part and the streptavidin column. The interaction between IFX Fc portion and streptavidin was examined using samples containing only detection reagents (FIGS. 11 and 12).

Hypothetically, samples containing only fluorescently labeled reagents will not result in any detection since neither capture reagent nor ADA molecules are present. In samples containing f-Fab only noise is measurable but in samples containing f-IFX background response is detectable. This unspecific interaction is only visible in Gyrolab if IFX is fluorescently labeled but not when IFX is biotinylated. This could be one explanation for the differences seen in background response.

There is a possibility that the difference in labeling procedures also could contribute to the explanation of differences in background response. Intact IFX are fluorescently labeled by conjugation at amine groups while Fab fragments are labeled at the hinge thiol that remains after reduction of F(ab′)₂. That is, f-Fab fragments are theoretically conjugated at one site while f-IFX may have several conjugations. Hypothetically, f-IFX would therefore result in higher detection signals compared to f-Fab.

Both biotinylation and fluorescent conjugation of Fab fragments at amine groups would have been interesting to evaluate.

### Evaluation of False Negative Reactions

When evaluating different assay variants one run where assay variant b-Fab and f-Fab and b-IFX and f-IFX where diluted in Rexxip™ ADA and neat serum where conducted (FIG. 3).

Fab fragments displayed higher signal responses than IFX when diluted in both neat serum and Rexxip™ ADA. Increased response dynamics confirms that this increase in signal intensity is not dependent on increased background response. One explanation for this could be that IFX reagents, due to bivalency, has greater tendency to form incomplete complexes and large complexes with anti-IFX compared to Fab fragments. Hypothetically it seems when the Fc part is removed the monovalent Fab fragments have lower tendency to form unwanted complexes with anti-IFX hence higher response signals are detected.

Further investigation of false negative reactions was conducted by comparison of four equimolar reagent concentrations for assay variant b-Fab and f-Fab and b-IFX and f-IFX (FIG. 4a-d). In all reagent concentrations Fab fragments showed (or are presumed to show) higher response signals than IFX, possibly caused by fewer false negative reactions. At high reagent concentrations (FIG. 4c-d) background response interferes with signal response resulting in decreased response dynamics. Higher standard points would probably provide a standard curve resembling the ones in FIG. 4a-b. These findings lend support to the conclusion that monovalent Fab fragments have lower tendency to form unwanted complexes compared to intact IFX.

### Evaluation of False Positive Reactions and Cut Point

### Screening Negative Samples

The determined CP for assay variant b-IFX and f-IFX were marginally lower (0.22) than for assay variant b-IFX and f-Fab (0.24). This means the chance of detecting low anti-IFX concentrations could be higher for the IFX assay compared to the Fab assay. But determined CP for the IFX assay resulted in two blood donor samples screened positive for anti-IFX. This could be an indication of too low CP level.

For a more accurate CP determination more samples preferably from patients qualified for IFX treatment, but not treated, should have been used.

### Screening Patient Samples

When screening IFX patient samples a majority of positive samples for both assays were not categorized definitely positive (average response values at least two times floating cut point), meaning at least one more screening would probably be necessary to confirm the incidence of anti-IFX.

All patient samples were taken before infusions of IFX. 8 of the 50 patient samples were taken before the first infusion of IFX was administrated, these patient samples should reasonably be expected to not contain anti-IFX. 3 of these patient samples were screened as positive (not definitely positive) in the Fab assay. If these samples were screened as positive in both assay variants there is a possibility patients could somehow have developed anti-IFX before treatment. But since none of these samples were screened positive in the IFX assay the Fab assay could display false positive reactions caused by other serum factors. Another explanation could be that the calculated CP for the Fab assay was too low or that these samples are within the limit of false positive reactions included in the statistical calculation.

### Evaluation of False Positive Reactions

In a bridging immunoassay the prevalence of RFs can interfere by bridging Fc portions of capture and detecting reagents, this unwanted interaction will increase signal responses and therefore cause false positive reactions (Araujo et al. 2011).

Of 19 positive RF samples available none were found positive in the Fab assay while 13 (68%) were found positive in the IFX assay. These findings demonstrate that RF interferes and causes false positive reactions in assay variants containing intact capture and detection reagents. Using fab fragments as detection reagents cause no interference since RFs only bind the intact capture reagent. These findings indicate that some of the patient samples screened positive in the IFX assay but not in the Fab assay could be false positive reactions caused by RF.

The positive RF samples were screened using a nephelometer. There is no correlation between RF values and average response values. Hypothetically, one possible explanation for this could be if samples contain different RF isotypes. RF is predominantly of isotype IgM but can also occur as IgG, IgA and IgE (Gioud-Paquet et al. 1987). The difference in avidity and affinity for the different isotypes could hypothetically affect signal intensities differently in Gyrolab than in measurements using a nephelometer.

Intact reagents have a greater tendency to aggregate and together with ADA form unwanted complexes which causes false negative reactions, compared to Fab reagents. This means, when compared to an assay based on Fab fragments, intact reagents display reduced signal intensity and decreased assay sensitivity. Also an assay based on intact reagents is affected by false positive reactions caused by RF. Fab reagents are also likely affected by serum factors, probably anti-hinge autoantibodies, which reduces assay sensitivity.

An assay based on the combination between intact capture reagent and Fab fragment as detection is not affected by false positive reactions caused by RF nor by anti-hinge autoantibodies. Findings indicate an assay based on b-intact IFX and fluorophore labeled IFX Fab fragments is more sensitive and less affected by false positive reactions caused by interference from RF, compared to an assay based on intact IFX reagents. Findings also indicate an assay based completely on IFX Fab fragments is less affected by false negative reactions hypothetically caused by unwanted complex formation, compared to an assay based completely on intact IFX reagents. Results from this project indicate ADA assay performance for therapeutic mAbs on Gyrolab may be improved by using an assay based partly on Fab reagents instead of intact reagents.

### Development of a Functioning Fab Fragment ADA Assay

### Drug Tolerance

Herein, drug tolerance of assay variants has been investigated by drug concentrations 8-500 μg/mL (FIG. 8).

In the experiment, 40 nM biotinylated Fab, 40 nM fluorophore labeled Fab, 20 nM biotinylated Infliximab and 20 nM fluorophore labeled Infliximab were used. Standard points, 125-4000 ng/mL anti-Infliximab diluted in neat serum spiked with free drug were measured in triplicates. Blank samples containing neat serum spiked with drug were measured in 6-plicate. Standard points and blank samples were diluted 1:10 in Rexxip™ ADA before analysis on Gyrolab Mixing™ CD with acid dissociation.

The results are shown in FIG. 8: a) Drug concentration b-Fab, f-Fab 8-500 μg/mL. Response on y-axis 0.1-100. b) Drug concentration b-Fab f-Intact 8-500 μg/mL. Response on y-axis 0.1-100 c) Drug concentration b-Intact f-Fab 8-500 μg/m L. Response on y-axis 0.1-100. d) Drug concentration b-Intact f-Intact 8-500 μg/mL. Response on y-axis 0.1-100.

The results indicate assay variant b-IFX and f-Fab is the most drug tolerant assay variant.

### Acid Dissociation

One issue when dissociating drug-ADA complexes using acidic buffers is inactivation of some ADAs (Li et al. 2011). One small experiment on the effects of acid dissociation was conducted using an assay based on Fab (FIG. 10).

The results are shown in FIG. 10. 40 nM biotinylated Fab and 40 nM fluorophore labeled Fab were used. Standard points, 125-4000 ng/mL anti-Infliximab diluted in neat serum spiked with 0-10 μg/mL free drug were measured in triplicates. Standard points were diluted 1:10 in Rexxip™ ADA before analysis on Gyrolab Mixing™ CD with acid and neutral dissociation.

Response dynamics is completely destroyed by presumed ADA-drug complexes at concentrations as low as 1 μg/mL when acidic dissociation is not used indicating the importance of acidic dissociation at least in the context of the method utilizing Gyrolab™.

### Gyros AB (2011). Gyrolab User Guide P0004354.

Gyros AB. (2014a). Optimized consumables. http://www.gyros.com/products/products-optimized/optimized-consumables/ [2014 May 26]
Gyros AB. (2014b). Gyrolab Bioaffy CDs. http://www.gyros.com/products/products-optimized/gyrolab-bioaffy-cds/ [2014 May 26]
Gyros AB. (2014c). Gyrolab Mixing CD. http://www.gyros.com/products/products-optimized/gyrolab-mixing-cd/ [2014 May 26]

### REFERENCES

Mikulskis A, Yeung D, Subramanyam M, Amaravadi L. Solution ELISA as a platform of choice for development of robust, drug tolerant immunogenicity assays in support of drug development. JIM, 2011, 365, 38-49
Reichert et al 2012, “Marketed therapeutic antibodies compendium” mAbs 4:3, 413-415; May/June
Reichert et al 2013, “Which are the antibodies to watch in 2013” mAbs 5:1, 1-4 January
Andrew, S M. & Titus, J A. (2000) Purification of Immunoglobulin G. Curr Protoc Cell Biol, 16.3.1-16-3-12. DOI: 10.1002/0471143030.cb1603s05.
Araujo, J., Zocher, M., Wallace, K., Peng, K. & Fischer, S K. (2011). Increased rheumatoid factor interference observed during immunogenicity assessment of an Fc-engineered therapeutic antibody. J Pharm Biomed Anal, 55 (5), pp. 1041-1049.
Bao, J., Yue, T., Li, T., He, D-Y. & Bao, Y-X. (2014). Good response to infliximab in rheumatoid arthritis following failure of interleukin-1 receptor antagonist. International Journal of Rheumatic Diseases. In press. DOI: 10.1111/1756-185X.12387.
Barry, R. & Ivanov, D. (2004). Microfluidics in biotechnology. J Nanobiotechnology, 2(1), pp. 2-7.
Berg, J M., Tymoczko, J L. & Stryer, L. (2002). Biochemistry. 5th ed. New York: W.H.Freeman.
Brerski, R J., Knight, D M. & Jordan, R E. (2011). The origins, specificity, and potential biological relevance of human anti-IgG hinge autoantibodies. Scientific World Journal, 11, pp. 1153-1167.
Corper, A L., Sohi, M K., Bonagura, V R., Steinitz, M., Jefferis, R., Feinstein, A., Beale, D., Taussig, M J. & Sutton, B J. (1997). Structure of human IgM rheumatoid factor Fab bound to its autoantigen IgG Fc reveals a novel topology of antibody-antigen interaction. Nat Struct Biol, 4(5), pp. 374-381.
Cox, K L., Devanatayan, V., Kriauciunas, A., Manetta, J., Montrose, C. & Sittampalam, S. (2012). Immunoassay Methods. Indianapolis: Eli Lilly & Company.
DeSilva, B., Smith, W., Weiner, R., Kelley, M., Smolec, J., Lee, B., Khan, M., Tacey, R., Hill, H. & Celniker, A. (2003). Recommendations for the bioanalytical method validation of ligand-binding assays to support pharmacokinetic assessments of macromolecules. Pharm Res, 20(11), pp. 1885-1900.
Drugbank (2014). 1,4-Dithiothreitol (accession number: DB04447) http://www.drugbank.ca/drugs/DB04447#pharmacology [2014 May 26]
Dudal, S., Baltrukonis, D., Crisino, R., Goyal, M J., Joyce, A., Osterlund, K., Smeraglia, J., Taniguchi, Y. & Yang, J. (2014). Assay formats: Recommendation for best practices and harmonization from the global bioanalysis consortium harmonization team. AAPS J, 16(2), pp. 194-205.
Food and Drug Administration (2014). Highlights of prescribing information Remicade (Reference ID: 3284123). U.S. Food and Drug Administration.
Gioud-Paquet, M., Auvinet, M., Raffin, T., Girard, P., Bouvier, M., Lejeune, E. & Monier, J C. (1987). IgM rheumatoid factor (RF), IgARF, IgERF and IgGRF detected by ELISA in rheumatoid arthritis. Ann Rheum Dis, 46(1), pp. 65-71.
Graphpad Software. (2014) Outlier calculator. http://graphpad.com/quickcalcs/Grubbs1.cfm [2014 May 26]
Grubbs, F E. (1969). Procedures for detecting outlying observations in sample. Technometrics, 11(1), pp. 1-21.
Hansel, T T., Kropshofer, H., Singer, T., Mitchell, J A. & George, A J. (2010). The safety and side effects of monoclonal antibodies. Nat Rev Drug Discov, 9(4), pp. 325-338.
Hermansson, G T. (1996). Bioconjugate techniques. San Diego: Academic Press.
Huh, J H., White, A J., Brych, S R. Franey, H. & Matsumura, M. (2013). The identification of free cysteine residues within antibodies and a potential role for free cysteine residues in covalent aggregation because of agitation stress. J Pharm Sci, 102(6), pp. 1701-1711.
Inganäs, M., Derand, H., Eckersten, A., Ekstrand, G., Honerud, A K., Jesson, G., Thorsen, G., Soderman, T. & Andersson, P. (2005) Integrated microfluidic compact disc device with potential use in both centralized and point-of-care laboratory settings. Clin Chem, 51(10), pp. 1985-1987.
Janeway C A., Travers P., Walport M. & Shlomchik, M J. (2001). Immunobiology: The immune system in health and disesase. 5th ed. New York: Garland Science.
Johansson, A., Erlandsson, A., Eriksson, D., Ullén, A., Holm, P., Sundström, B E., Roux, K H. & Stigbrand, T. (2002). Idiotypic-anti-idiotypic complexes and their in vivo metabolism. Cancer, 94(4), pp. 1306-1313.
Joyce, A P., Wang, M., Lawrence-Henderson, R., Filliettaz, C., Leung, S S., Xu, X. & O'Hara, D M. (2014). One Mouse, One Pharmacokinetic Profile: Quantitative Whole Blood Serial Sampling for Biotherapeutics. Pharm Res.
Kan, K S., Anderson, V A., Leong, W S., Smith, A M., Worth, A T. & Stevenson, G T. (2001). Thioether-bonded constructs of Fab′ gamma and Fc gamma modules utilizing differential reduction of interchain disulfide bonds. J Immunol, 166(2), pp. 1320-1326.
Kato, K., Umeda, U., Suzuki, F., Hayashi, D. & Kosaka A. (1979). Use of antibody Fab′ fragments to remove interference by Rheumatoid factors with the enzyme-linked sandwich immunoassay. FEBS letters, 102(2), pp. 253-256.
Keizer, R J., Huitema, A D R., Schellens, J H M. & Beijnen, J H. (2010). Clinical Pharmacokinetics of therapeutic monoclonal antibodies. Clin Pharmacokinet, 49(8), pp. 493-507.
Köhler, G. & Milstein, C. (1975). Continuous cultures of fused cells secreting antibody of predefined specificity. Nature, 256(5517), pp. 495-497.
Lee, W., Oh, B-K., Lee W H. & Choi J-W. (2005). Immobilization of antibody fragment for immunosensor application based on surface plasmon resonance. (2005). Colloids Surf B Biointerfaces, 40 (3/4), pp. 143-149.
Li, J., Schantz, A., Schwegler, M. & Shankar, G. (2011). Detection of low-affinity anti-drug antibodies and improved drug tolerance in immunogenicity testing by Octet biolayer interferometry. J Pharm Biomed Anal, 54 (2), pp. 286-294.
Liu, X F., Wang, X., Weaver, R J., Calliste, L., Xia, C., He, Y J. & Chen, L. Validation of a Gyrolab™ assay for quantification of rituximab in human serum. J Pharmacol Toxicol Methods, 65(3), pp. 107-114.
Lodish, H., Berk, A., Zipursky S L., Matsudaira, P., Baltimore, D. & Darnell, J. (2000). Molecular cell biology. 4th ed. New York: W.H.Freeman.
Lonberg, N. (2005). Human antibodies from transgenic animals. Nat Biotechnol, 23(9), pp. 1117-1125.
Mikulskis, A., Yeung, D., Subramanyam, M. & Amaravadi, L. (2011) Solution ELISA as a platform of choice for development of robust, drug tolerant immunogenicity assays in support of drug development. J Immunol Methods, 365(1-2), pp. 38-49.
Nishi, H., Miyajoma, M., Wakiyama, N., Kubota, K., Hasegawa, J., Uchiyama, S. & Fukui, K. (2011). Fc domain mediated self-association of an IgG1 monoclonal antibody under a low ionic strength condition. J Biosci Bioeng, 112(4), pp. 326-332.
Peters, B J., Capelle, M A., Arvinte, T. & van de Garde, E M. (2013). Validation of an automated method for compounding monoclonal antibody patient doses: case studies of Avastin (bevacizumab), Remicade (infliximab) and Herceptin (trastuzumab). MAbs, 5(1), pp. 162-170.
Rispens, T., de Vrieze, H., de Groot, E., Wouters, D., Stapel, S., Wolbink, G J. & Aarden, L A. (2012). Antibodies to constant domains of therapeutic monoclonal antibodies: Anti-Hinge antibodies in immunogenicity testing. J Immunol Methods, 375(1/2), pp. 93-99.
Shankar, G., Devanarayan, V., Amaravadi, L., Barrett, Y C., Bowsher, R., Finco-Kent, D., Fiscella, M., Gorovits, B., Kirschner, S., Moxness, M., Parish, T., Quarmby, V., Smith, H., Smith, W., Zuckerman, L A. & Koren, E. (2008). Recommendations for the validation of immunoassays used for detection of host antibodies against biotechnology products. J Pharm Biomed Anal, 48(5), pp. 1267-1281.
Shapiro, S S. & Wilk, M B. (1965). An analysis of variance test for normality (complete samples). Biometrika, 52(3/4), pp. 591-611.
Shapiro-Wilk normality test. (2014) http://sdittami.altervista.org/shapirotest/ShapiroTest.html [2014 May 26]
Sloan, J H., Ackermann, B L., Carpenter, J W., Nguyen, H T., Stopa, K., Siegel, R W. & Konrad, R J. (2012). A novel high-sensitivity and drug-tolerant sandwich immunoassay for the quantitative measurement of circulating proteins. Bioanalysis, 4(3), pp. 241-248.
St Clair, E W., Wagner, C L., Fasanmade, A A., Wang, B., Schaible, T., Kavanaugh, A. & Keystone, E C. (2002). The relationship of serum infliximab concentrations to clinical improvement in rheumatoid arthritis: results from ATTRACT, a multicenter, randomized, double-blind, placebo-controlled trial. Arthrtis Rheum, 46(6), pp. 1451-1459.
Tatarewicz, S., Miller J M., Swanson, Sj. & Moxness, M S. (2010). Rheumatoid factor interference in immunogenicity assays for human monoclonal antibody therapeutics. J Immunol Methods, 357(1/2), pp. 10-16.
Wild, D G. (2013). The immunoassay handbook: theory and applications of ligand binding, ELISA and related techniques. 4th ed. Oxford: Elsevier.

1. An in vitro method for the detection of an anti-drug antibody (ADA) against a therapeutic drug antibody (TDA) in a biological sample, said method comprising:
capturing and attaching said ADA onto a solid phase via a capture reagent (1), said capture reagent comprising an affinity moiety (1a) for said ADA and a capturing moiety (1b) for attachment to a solid phase; and
detecting ADA with a detection reagent (2), said detection reagent comprising an affinity moiety (2a) for said ADA and a detection moiety (2b) for detecting the presence of ADA in the sample, wherein said capture reagent (1) and said detection reagent (2) thereby forms a bridging complex with said ADA,
wherein the method is characterized in that
said affinity moiety (1a) of said capture reagent (1) and said affinity moiety (2a) of said detection reagent (2) are selected from the group consisting of an intact TDA and one or more Fab fragment(s) of said TDA; and
a) when the affinity moiety (1a) of said capture reagent (1) is an intact TDA, the affinity moiety (2a) of said detection reagent (2) is one or more Fab fragment(s) of said TDA, and
b) when the affinity moiety (1a) of said capture reagent (1) is one or more Fab fragment(s) of said intact TDA, the affinity moiety (2a) of said detection reagent (2) is an intact TDA.

2. An in vitro method according to claim 1, wherein the method comprises the steps of:
a) simultaneously contacting the biological sample with said capture reagent (1) and said detection reagent (2) to obtain a reaction mixture,
b) adding the reaction mixture of step a) to a solid phase allowing any bridging complex formed in step a) to attach thereto, and thereafter
c) detecting any ADA from said biological sample.

3. An in vitro method according to claim 1, wherein the method comprises the steps of:
a) adding the capture reagent (1) to the solid phase allowing the capture reagent (1) to attach thereto,
b) mixing the detection reagent (2) and the biological sample to obtain a reaction mixture,
c) adding the reaction mixture obtained in step b) to the solid phase of step a) allowing any ADA present in said reaction mixture to attach to the solid phase, and thereafter
d) detecting any ADA from said biological sample.

4. An in vitro method according to claim 3, wherein step a) is preceded by a step of adding to and incubating said biological sample with an acidic buffer before the biological sample is contacted with the capture reagent (1) and the detection reagent (2) said step being followed by a neutralization of said biological sample before step a) is performed.

5. An in vitro method according to claim 1, wherein the affinity moiety (1a) of the capture reagent (1) is an intact TDA and the affinity moiety (2a) of the detection reagent (2) is one or more Fab fragment(s) of said intact TDA.

6. An in vitro method according to claim 1, wherein the affinity moiety (1a) of the capture reagent (1) is one or more Fab fragment(s) of said intact TDA and the affinity moiety (2a) of the detection reagent (2) is an intact TDA.

7. An in vitro method according to claim 1, wherein said TDA is an intact antibody providing for cleavage thereof thereby generating one or more Fab fragment(s) from the intact antibody.

8. An in vitro method according to claim 1, wherein said therapeutic antibody may be an antibody having affinity for TNF, such as TNF-α.

9. An in vitro method according to claim 1, wherein said TDA is selected from the group consisting of: Infliximab, Adalimumab, Bevacizumab and Denosumab.

10. An in vitro method according to claim 1, wherein the TDA is Inflimixab.

11. An in vitro method according to claim 1, wherein the capturing moiety (1b) of said capture reagent (1) is biotin and the solid phase comprises streptavidin.

12. An in vitro method according to claim 1, wherein the detection moiety (2b) comprises a luminescent label selected from the group consisting of a fluorescent label, a phosphorescent label, and a radioluminescent label.

13. An in vitro method according to claim 12, wherein the label is selected from the group consisting of: Alexa Fluor 633, Alexa Fluor 647, Dylight 649, Dylight 650, Cy-5, Cy-5.5, CF 647 Dye, Innova 791-0010 and fluorescent nanoparticles.

14. An in vitro method according to claim 1, wherein the biological sample comprises whole blood, serum or plasma.

15. An in vitro method according to claim 1, for determining if subject treated with a therapeutic drug antibody has developed a sensitivity or an adverse drug reaction against said therapeutic drug antibody.

16. A kit for performing an in vitro method according to claim 1, wherein said kit comprises:
a) a capture reagent (1) having an affinity moiety which comprises an intact TDA and a detection reagent (2) having an affinity moiety (2a) which comprises one or more Fab fragment(s) of said TDA, or
b) a capture reagent (1) having an affinity moiety (1b) which comprises one or more Fab fragment(s) of a TDA and a detection reagent (2) comprising an intact TDA, and optionally instructions for use.

17. The in vitro method according to claim 1, wherein the affinity moiety of the capture reagent is an intact antibody and the affinity moiety of the detection reagent is a monovalent Fab fragment of the TDA.

18. The in vitro method according to claim 1, wherein the TDA is an intact IgG antibody which allows cleavage in the hinge region of the antibody.
